SEQUENCE LISTING (10 sequences)

SEQ ID NO: 1 - DNA, 538 bp, herpes simplex virus
  source 1..538: mol_type = genomic DNA
cgtcacatccaggccggcggaaaccggaacggcatatgcaaattggaaactgtcctgtcttggggcccacccacccgacgcgtcatatgcaaatgaaaatcggtcccccgaggccacgtgtagcctggatcccaacgaccccgcccatgggtcccaattggccgtcccgttaccaagaccaacccagccagcatatccacccccgcccgggtccccgcggaagcggaacggtgtatgtgatatgctaattaaatacatgccacgtacttatggtgtctgattggtccttgtctgtgccggaggtggggcgggggccccgcccggggggcggaacgaggaggggtttgggagagccggccccggcaccacgggtataaggacatccaccacccggccggtggtggtgtgcagccgtgttccaaccacggtcacgcttcggtgcctctccccgattcgggcccggtcgctcgctaccggtgcgccaccaccagaggccatatccgacaccccagccccgacggcagccgacagcccggtc

SEQ ID NO: 2 - DNA, 20 bp, Homo sapiens
  source 1..20: mol_type = genomic DNA
ggcattcaccgcgtgcctta

SEQ ID NO: 3 - DNA, 22 bp, Homo sapiens
  source 1..22: mol_type = genomic DNA
tgagctacagtgcttcatctca

SEQ ID NO: 4 - protein, 554 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 5 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 6 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 7 - protein, 19 aa, Homo sapiens
ATNFSLLKQAGDVEENPGP

SEQ ID NO: 8 - DNA, 279 bp, Homo sapiens
  source 1..279: mol_type = genomic DNA
ttaccgaccacccgcaaacagcagggtcccctgggcttcccaagccgcgcacctctccgccccgcccctgcgccctccttcctcgcgtctgcccctctcccccaccccgccttctccctccccgccccagcggcgcatgcgccgcgctcggagcgtgtttttataaaagtccggccgcggccagaaacttcagtttgttggctgcggcagcaggtagcaaagtgacgccgagggcctgagtgctccagtagccaccgcatctggagaaccagcggttac

SEQ ID NO: 9 - DNA, 22 bp, Homo sapiens
  source 1..22: mol_type = genomic DNA
tggggtatttgacaaactgaca

SEQ ID NO: 10 - DNA, 22 bp, Homo sapiens
  source 1..22: mol_type = genomic DNA
tcacaagttagggtctcaggga

(19) United States
(12) Patent Application Publication
(10) Pub. No.: US 2025/0161384 A1
(43) Pub. Date: May 22, 2025

(54) TRANSCRIPTIONAL AND TRANSLATIONAL DUAL REGULATED ONCOLYTIC HERPES SIMPLEX VIRUS VECTORS

(71) Applicants: Virogin Biotech (Shanghai) Ltd, Shanghai (CN); VIROGIN BIOTECH CANADA LTD, Richmond (CA)

(72) Inventors: William Wei-Guo Jia, Vancouver (CA); Jun Ding, Vancouver (CA); Guoyu Liu, Vancouver (CA); Xiaohu Liu, Vancouver (CA); Dmitry V. Chouljenko, Vancouver (CA)

(21) Appl. No.: 18/730,261

(22) PCT Filed: Jan. 30, 2023
(86) PCT No.: PCT/US2023/061605

§ 371 Date: Jul. 18, 2024

Related U.S. Application Data
(60) Provisional application No. 63/304,584, filed on Jan. 29, 2022.

(51) Int. Cl. A61K 35/766; A61P 35/00; A61P 37/04; C07K 14/54; C12N 7/00

(52) U.S. Cl. CPC A61K 35/766; A61P 35/00; A61P 37/04; C07K 14/5434; C07K 14/5443; C12N 7/00; C12N 2710/16622; C12N 2710/16632; C12N 2710/16643; C12N 2710/16662; C12N 2710/16671; C12N 2830/008; C12N 2840/007

(57) ABSTRACT

An HSV vector with ICP27 under control of CXCR4 promoter and ICP34.5 under control of miRNA-124/143, wherein the vector has a deletion of one RL and one RS region of the viral genome. Within optional embodiments the mutation is a deletion containing the second copy of the ICP34.5 gene. These constructs provide increased safety without sacrificing efficacy. The HSV vector also incorporates a virus-expressed cytokine cassette encoding IL-12, IL-15/IL-15RA under the control of CMV promoter.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD OF THE INVENTION

The present invention relates generally to oncolytic herpes simplex virus (oHSV) vectors that express molecules that stimulate the immune system.

### REFERENCE TO SEQUENCE LISTING, TABLE OR COMPUTER PROGRAM

The contents of the electronic sequence listing (VIRO_416PC_SeqList.text.xml; Size: 10.9 bytes; and Date of Creation: Jan. 29, 2023) is herein incorporated by reference in its entirety.

### BACKGROUND

Malignant tumors are a serious threat to human life and health. Although a variety of standard treatment options exist, such as surgery, radiotherapy, chemotherapy, targeted therapy, and immunotherapy (including immune checkpoint inhibitors), most patients with advanced tumors still have poor prognosis. At present, tumor immunotherapy has made breakthrough progress in the treatment of tumors. Immune-targeted drug therapy (e.g., immune checkpoint suppression) and immune cell therapy (e.g., chimeric antigen receptor T-cell (CAR-T)) have triggered changes in the field of anti-tumor therapy. However, among the currently approved indications for checkpoint inhibitors, the single-drug effective rate is only about 30% (Jiang et al., 2020, Progress and Challenges in Precise Treatment of Tumors With PD-1/PD-L1 Blockade. Frontiers in Immunology, 11 (March)); while CAR-T therapy mainly only targets Cluster of Differentiation 19 (CD19) and B cell maturation antigen (BCMA) that are highly expressed by B cell tumors. The clinical effectiveness in solid tumors has yet to be confirmed (Long et al., 2018, CAR T Cell Therapy of Non-hematopoietic Malignancies: Detours on the Road to Clinical Success. Frontiers in Immunology, 9 (December), 2740). There are still many malignant tumors where there is clear long-term evidence as to the benefits of immunotherapy.

There is no clinically effective treatment for malignant tumors relapsed after and refractory to standard treatment, and patients with this condition are likely to die sooner due to the extensive tumor metastasis or invasion of important organs. Therefore, these patients have an extremely high unmet need for effective treatment, leading to an urgent need to develop new treatment methods to control the progression of the disease and prolong the survival of patients.

The present invention overcomes shortcomings of current cancer therapies, including immunotherapies, and further provides additional unexpected benefits.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

Briefly stated, the invention relates to compositions and methods for treating cancer with recombinant herpes simplex virus vectors. Within one embodiment of the invention recombinant herpes simplex viruses are provided comprising a modified oncolytic herpes virus genome, wherein the modified herpes virus genome comprises at least one miRNA target sequence operably linked to a first copy of an ICP34.5 gene, and a second copy of the ICP34.5 gene comprises an inactivating mutation. Within various embodiments, the recombinant virus can comprise from one to ten miRNA target sequences operably linked to the first copy of the ICP34.5 gene.

Within further embodiments, the miRNA target sequences can bind at least two different miRNAs (e.g., one or more of miR-124, miR-124*, and miR-143).). Within certain embodiments the miR target sequences include SEQ ID NO. 2 (a miR-124 binding sequence); SEQ ID NO. 3 (a miR-143 binding sequence); SEQ ID No. 9 (a miR-223 binding sequence); and/or SEQ ID NO. 10 (a miR-125b binding sequence). Other miRNA target sequences that can be utilized within other embodiments of the invention include, for example, mIR-122, miR-127, miR-128, miR-129, miR-129*, miR-132, mIR-133a, mIR133b, miR-135b, miR-136, miR-136*, miR-137, miR-139-5p, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-539, miR-541, miR-543*, miR-551b, miR-758, and miR-873 as described in WO2020/113151, which is incorporated by reference in its entirety.

Within yet other embodiments, the recombinant herpes virus can further comprise at least one nucleic acid encoding a non-viral protein. Examples of non-viral proteins include immunostimulatory factors, antibodies, and checkpoint blocking peptides (e.g., a peptide derived from PD-1 or from PD-L1 or PD-L2, or, an antibody against a checkpoint inhibitor such as anti-PD-1, anti-PD-L1, anti-PD-L2, or, anti-CTLA-4), wherein the at least one nucleic acid is operably linked to a strong promoter (e.g., a viral promoter such as CMV or other promoter such as EF-1alpha or CAG). Alternatively, the at least one nucleic acid is operably linked to a tumor-specific or tumor-associated promoter (e.g., CEA promoter, CXCR4 promoter, hTERT promoter, Survivin promoter, BRMS1 promoter, MCM5 promoter, or RAN promoter). Within particularly preferred embodiments, the non-viral protein is one, or all of IL12, IL15, IL15 receptor alpha subunit. Representative examples of these sequences include SEQ ID NO. 4 (IL12); SEQ ID NO. 5 (IL15); and SEQ ID NO. 6 (IL15 receptor alpha subunit).

Within yet other embodiments the recombinant herpes simplex virus further comprises a nucleic acid sequence encoding a glycoprotein with enhanced fusogenicity (as compared to a similar wild-type virus). Examples include a wide variety of transgenes (e.g., a fusogenic glycoprotein from Gibbon Ape Leukemia Virus “GALV”), and/or mutations which enhance HSV fusion, including for example, truncations or mutations in glycoprotein B, glycoprotein K, and or UL20.

In yet other embodiments, the recombinant herpes virus can further comprise a ICP27 promoter sequence operably linked to a first copy of an ICP47 gene, wherein the natural promoter of the ICP47 gene comprises an inactivating deletion.

Within yet other embodiments, the recombinant herpes virus can further comprise an inactivating mutation in at least one copy of an ICP4 gene or an ICP0 gene.

In yet other embodiments, the recombinant herpes virus can further comprise a tumor-specific promoter operably linked to a first copy of an ICP27 gene, wherein the natural ICP27 promoter comprises an inactivating mutation.

Also provided are therapeutic compositions comprising the recombinant herpes viruses described herein, as well as methods of lysing tumor cells, and, methods of treating cancers in a subject comprising the step of administering one of the recombinant herpes viruses described herein to a subject.

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one exemplary embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

### BRIEF DESCRIPTION OF THE DRAWINGS

Exemplary features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 diagrammatically depicts a Transcription and Translation Dual Regulated (“TTDR”) system as exemplified by the overall structural organization of the double-stranded deoxyribonucleic acid (DNA) elements of VG2062 (also referred to as VG203).

FIG. 2 is a graph (top panels) and table (bottom panel) demonstrating microRNA-mediated control of viral gene expression.

FIG. 3 is a comparison of cell-to-cell fusion phenotypes between three recombinant oncolytic HSVs.

FIG. 4 depicts qPCR-based quantification of VG2062 ICP27 DNA and host cell CXCR4 mRNA in 9 different urological tumor cell lines.

FIG. 5 depicts replication kinetics of VG2062 on a panel of 9 different urological tumor cell lines.

FIG. 6 depicts IC50 cytotoxicity values (measured in MOI) for VG2062 and VG161 on a panel of 10 different urological tumor cell lines.

FIG. 7 depicts ELISA-based quantification of human IL-12 and human IL-15 payloads secreted by VG2062 and VG161 after infecting a panel of 10 different urological tumor cell lines.

FIG. 8 depicts tumor volumes from A549 tumor-bearing immunocompromised mice that were treated with three-dose injection of VG2062.

FIG. 9 depicts tumor volumes from PC tumor-bearing immunocompromised mice that were treated with escalating single doses of VG2062.

FIG. 10 depicts tumor volumes from DU145 tumor-bearing immunocompromised mice that were treated with escalating single doses of VG2062.

FIG. 11 depicts tumor volumes from UM-UC-3 tumor-bearing immunocompromised mice that were treated with either low dose or high dose single injection of VG2062.

FIG. 12 depicts tumor volumes from BT-474 tumor-bearing immunocompromised mice that were treated with either low dose or high dose single injection of VG2062.

FIG. 13 depicts tumor volumes from EMT-6 tumor-bearing immunocompetent mice that were treated with either low dose or high dose triple-injection of mVG2031.

FIG. 14 depicts tumor volumes from EMT-6 tumor-bearing immunocompetent mice that were treated with mVG2031, with anti-PD-1 antibody, or with a combination of both mVG2031 and anti-PD-1 antibody.

FIG. 15 depicts tumor volumes from Renca tumor-bearing immunocompetent mice that were treated with either low dose single injection of mVG2031, high dose single injection of mVG2031, low dose triple-injection of mVG2031, or high dose triple-injection of mVG2031.

FIGS. 16A and 16B depicts tumor volumes from immunocompetent mice that were bilaterally implanted with Renca tumors on both the left (FIG. 16B) and right (FIG. 16A) flanks and treated with mVG2031, with anti-PD-1 antibody, or with a combination of both mVG2031 and anti-PD-1 antibody.

FIG. 17 depicts tumor volumes from A-498 tumor-bearing immunocompromised mice that were treated with either low dose or high dose single injection of VG2062.

FIG. 18 depicts mouse eyes after corneal scarification and inoculation with either wild-type HSV-1 strain 17+ or with either low dose or high dose VG2062.

### DETAILED DESCRIPTION OF THE INVENTION

The term “oncolytic herpes virus” or “oHSV” refers generally to a herpes virus capable of replicating in and killing tumor cells. Within certain embodiments the virus can be engineered in order to more selectively target tumor cells. Representative examples of oncolytic herpes viruses are described in U.S. Pat. Nos. 7,223,593, 7,537,924, 7,063,835, 7,063,851, 7,118,755, 8,216,564, 8,277,818, and 8,680,068, all of which are incorporated by reference in their entirety.

“Treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemia's, lymphomas, myelomas and sarcomas. Further examples include, but are not limited to cancer of the bile duct cancer, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, larynx, lung, liver, oral cavity, ovaries, pancreas, prostate, skin (e.g., melanoma and squamous cell carcinoma) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy).

Particularly preferred cancers to be treated include lung tumors, breast and prostate tumors, glioblastomas, urological tumors, tumors of the gastro-intestinal tract (and associated organs) e.g., esophagus, cholangiocarcinoma, anal, stomach, bladder, kidney, intestine, pancreatic, colon and liver, and all surface injectable tumors (e.g., melanomas). Benign tumors and other conditions of unwanted cell proliferation may also be treated. More preferably, the cancer to be treated expresses a detectable amount of C-X-C Motif Chemokine Receptor 4 (CXCR4).

In order to further an understanding of the various embodiments herein, the following sections are provided which describe various embodiments: A. Oncolytic Herpes Viruses; B. Specific Herpes Virus Constructs—VG2062; C. Post-transcriptional Regulation; D. Expression of ICP27 in VG2062 is Transcriptionally Controlled; E. Payload Expression in VG2062 is Enhanced; F. Truncated Glycoprotein B; G. Modified ICP47 Promoter; H. Therapeutic Compositions; and I. Administration.

### A. Oncolytic Herpes Viruses

Herpes Simplex Virus (HSV) 1 and 2 are members of the Herpesviridae family, which infects humans. The HSV genome contains two unique regions, which are known as the unique long (U_{L}) and unique short (U_{S}) region. Each of these regions is flanked by a pair of identical inverted repeat sequences, with the repeats flanking the U_{L} region designated as R_{L} and the repeats flanking the U_{S} region designated as R_{S}. There are about 75 known open reading frames. The viral genome has been engineered to develop oncolytic viruses for use in e.g. cancer therapy. Tumor-selective replication of HSV may be conferred by mutation of the HSV ICP34.5 (also called γ34.5) gene. HSV contains two copies of ICP34.5. Mutants inactivating one or both copies of the ICP34.5 gene are known to lack neurovirulence, i.e. be avirulent/non-neurovirulent and be oncolytic. Tumor selective replication of HSV may also be conferred by controlling expression of key viral genes such as ICP27 and/or ICP4.

Suitable oncolytic HSV may be derived from either HSV-1 or HSV-2, including any laboratory strain or clinical isolate. In some embodiments, the oHSV may be derived from one of laboratory strains HSV-1 strain 17, HSV-1 strain F, or HSV-2 strain HG52. In other embodiments, it may be derived from non-laboratory strain JS-1. Other suitable HSV-1 viruses include HrrR3 (Goldstein and Weller, J. Virol. 62, 196-205, 1988), G2O7 (Mineta et al. Nature Medicine. 1(9):938-943, 1995; Kooby et al. The FASEB Journal, 13(11):1325-1334, 1999); G47Delta (Todo et al. Proceedings of the National Academy of Sciences. 2001; 98(11):6396-6401); HSV 1716 (Mace et al. Head & Neck, 2008; 30(8):1045-1051; Harrow et al. Gene Therapy. 2004; 11(22):1648-1658); HF10 (Nakao et al. Cancer Gene Therapy. 2011; 18(3):167-175); NV1020 (Fong et al. Molecular Therapy, 2009; 17(2):389-394); T-VEC (Andtbacka et al. Journal of Clinical Oncology, 2015: 33(25):2780-8); J100 (Gaston et al. PloS one, 2013; 8(11):e81768); M002 (Parker et al. Proceedings of the National Academy of Sciences, 2000; 97(5):2208-2213); NV1042 (Passer et al. Cancer Gene Therapy. 2013; 20(1):17-24); G207-IL2 (Carew et al. Molecular Therapy, 2001; 4(3):250-256); rQNestin34.5 (Kambara et al. Cancer Research, 2005; 65(7):2832-2839); G47Δ-mIL-18 (Fukuhara et al. Cancer Research, 2005; 65(23):10663-10668); and those vectors which are disclosed in PCT applications PCT/US2017/030308 entitled “HSV Vectors with Enhanced Replication in Cancer Cells”, and PCT/US2017/018539 entitled “Compositions and Methods of Using Stat1/3 Inhibitors with Oncolytic Herpes Virus”, all of the above of which are incorporated by reference in their entirety.

The oHSV vector has at least one γ34.5 gene that is modified with miRNA target sequences in its 3′ UTR as disclosed herein; there are no unmodified γ34.5 genes in the vector. In some embodiments, the oHSV has two modified γ34.5 genes; in other embodiments, the oHSV has only one γ34.5 gene, and it is modified. In some embodiments, the modified γ34.5 gene(s) are constructed in vitro and inserted into the oHSV vector as replacements for the viral gene(s). When the modified γ34.5 gene is a replacement of only one γ34.5 gene, the other γ34.5 is deleted. Either native γ34.5 gene can be deleted. In one embodiment, the terminal repeat region, which comprises γ34.5 gene and ICP4 gene, is deleted. Alternatively, the internal repeat region, which also comprises γ34.5 gene and ICP4 gene, is deleted. As discussed herein, the modified γ34.5 gene may comprise additional changes, such as having an exogenous promoter.

The oHSV may have additional mutations, which may include disabling mutations e.g., deletions, substitutions, insertions), which may affect the virulence of the virus or its ability to replicate. For example, mutations may be made in any one or more of ICP6, ICPO, ICP4, ICP27, ICP47, ICP24, ICP56. Preferably, a mutation in one of these genes (optionally in both copies of the gene where appropriate) leads to an inability (or reduction of the ability) of the HSV to express the corresponding functional polypeptide. In some embodiments, the promoter of a viral gene may be substituted with a promoter that is selectively active in target cells or inducible upon delivery of an inducer or inducible upon a cellular event or particular environment.

The oHSV may have additional mutations, which may include disabling mutations e.g., deletions, substitutions, insertions), which may reduce the risk of recombination events in a repetitive and non-unique region of the viral genome. For example, mutations may be made in any one or more of R_{S} region, R_{L} region, US1 promoter, US12 promoter. In some embodiments, the promoter of a viral gene may be substituted with a promoter of a different viral gene that reduces the risk of recombination event in the repetitive and non-unique region of the native, or natural, promoter.

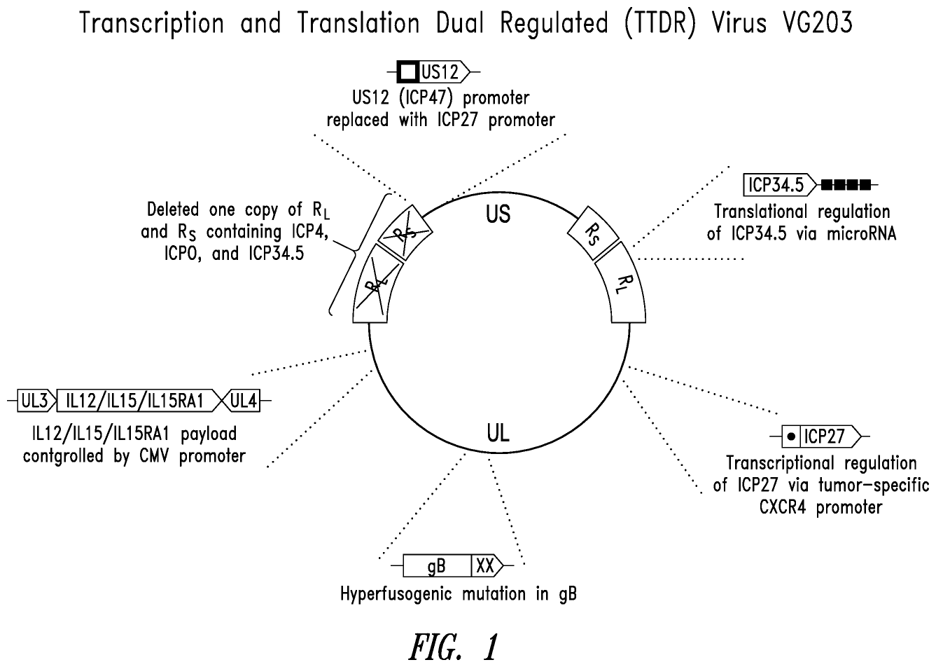

In certain embodiments, the expression of ICP4 or ICP27 is controlled by an exogenous promoter, e.g., a tumor-specific promoter. Exemplary tumor-specific promoters include survivin, CEA, CXCR4, PSA, ARR2PB, hTERT, RAN, BRMS1, MCM5, or telomerase; other suitable tumor-specific promoters may be specific to a single tumor type and are known in the art. Other elements may be present. In some cases, an enhancer such as NFkB/oct4/sox2 enhancer is present. As well, the 5′UTR may be exogenous, such as a 5′UTR from growth factor genes such as FGF. See FIG. 1 for an exemplary construct. See also SEQ ID NO. 8 for a representative sequence of a CXCR4 promoter.

The oHSV may also have genes and nucleotide sequences that are non-HSV in origin. For example, a sequence that encodes a prodrug, a sequence that encodes a cytokine or other immune stimulating factor, a tumor-specific promoter, a checkpoint inhibitor, an inducible promoter, an enhancer, a sequence homologous to a host cell, among others may be in the oHSV genome. Exemplary sequences encode IL12, IL15, IL15 receptor alpha subunit, OX40L, CTLA-4 blocker, PD-L1 blocker or a PD-1 blocker. For sequences that encode a product, they are operatively linked to a promoter sequence and other regulatory sequences (e.g., enhancer, polyadenylation signal sequence) necessary or desirable for expression.

Within certain embodiments of the invention, the oHSV expresses a PD-1 blocker, a PD-L1 blocker, or a PD-L2 blocker, e.g., a protein or peptide sequence that, when expressed, blocks, binds to, or otherwise inhibits PD-1, PD-L1, PD-L2, or a ligand thereof. Briefly, PD-1 (or “Programmed cell death protein 1”, and also known as CD279) is a protein on the surface of T and B cells that has a role in down regulating the immune system, thereby suppressing T cell inflammatory activity and preventing or decreasing the ability of the immune system to kill tumor cells.

PD-1 has two ligands: PD-L1 and PD-L2. Representative examples of PD-1, PD-L1 and PD-L2 antagonists include peptides and antibodies that block, bind to or otherwise inhibits the binding of PD-1 or its ligands in a statistically significant manner, thereby inhibiting or diminishing their activity. Representative examples of PD-L1 antagonists include nucleic acid sequences that encode any of the following: Atezolizumab (TECENTRIQ—a humanized anti-PD-L1 antibody); Avelumab (BAVENCIO—a fully human anti-PD-L1 antibody); Durvalumab (IMFINZI—a human anti-PD-L1 antibody); Durvalumab (IMFINZI—a fully human igG1 antibody); KN035; Cosibelimab (CK-301 by Checkpoint Therapeutics; AUNP12 (a peptide PD-1/PD-L1 inhibitor by Aurigene and Laboratoires Pierre Fabre); CA-170; and BMC-986189 (a macrocyclic peptide).

Representative examples of PD-1 antagonists include nucleic acid sequences that encode any of the following representative anti-PD-1 antibodies: Nivolumab (OPDIVO—a human anti-PD-1 antibody); Pembrolizumab (KEYTRUDA—a humanized anti-PD-1 antibody); Cemiplimab (LIBTAYO—an anti-PD-1 antibody); Dostarlimab (JEMPERLI—an anti-PD-1 antibody; Vopratelimab (JTX-4014) by Jounce Therapeutics; Spartalizumab (PDR001) by Novartis; Camrelizumab (SHR1210)—an anti-PD-1 monoclonal antibody by Jiangsu HengRui Medicine Co., Ltd.; Sintilimab (IBI308), a human anti-PD-1 antibody developed by Innovent and Eli Lilly; Tislelizumab (BGB-A317)—a humanized IgG4 anti-PD-1 monoclonal antibody; Toripalimab (JS 001) a humanized IgG4 monoclonal antibody against PD-1; INCMGA00012 (MGA012) a humanized IgG4 monoclonal antibody developed by Incyte and MacroGenics; AMP-224 by AstraZeneca/MedImmune and GlaxoSmithKline; and AMP-514 (MEDI0680) by AstraZeneca.

Representative patents and patent applications describing PD-1, PD-L1 and PD-L2 antagonists, methods for assessing their activity, and nucleic acid sequences that encode them include, for example: U.S. Pat. Nos. 7,595,048, 7,943,743, 8,952,136, 8,217,149, 8,609,089, 8,735,553, 8,779,105, 8,779,108, 8,993,731, and 9,815,897; U.S. Publication Nos. 2010/0203056, 2010/0266617, 2011/0229461, 2013/0017199, 2014/341917, 2015/0203579, 2016/0311903, 2016/0376367, 2016/0311903, 2017/0044259 and 2018/0346569; and PCT Publication No. WO2012145493; all of which are incorporated by reference in their entirety.

Within other embodiments of the invention the oHSV expresses a nucleic acid sequence encoding a CTLA-4 antagonist. Briefly, CTLA-4 also has two primary ligands: CD80 and CD86 on the surface of T cells. Representative examples of CTLA-4 antagonists include peptides and antibodies that block, bind to or otherwise inhibits the binding of CTLA-4 or it's ligands in a statistically significant manner, thereby inhibiting or diminishing their activity. Representative examples of CTLA-4 antagonists include: Ipilimumab (Yervoy—a fully human anti-CTLA-4 monoclonal antibody, IgG1 isotype); and Tremelimumab (Imjudo—a fully human anti-CTLA-4 antibody, IgG2 isotype). Representative examples of patents that relate to nucleic acid sequences that encode anti-CTLA-4 antibodies and variants thereof include U.S. Pat. Nos. 5,811,097, 5,855,887, 5,977,318, 6,051,227, 6,207,156, 6,682,736, 6,984,720, 7,109,003, 7,132,281 and 10,196,445, and PCT Publication Nos. WO 2001/014424, WO 2004/035607, 2005/0201994, all of which are incorporate by reference in their entirety.

The regulatory region of viral genes may be modified to comprise response elements that affect expression. Exemplary response elements include response elements for NF-κB, Oct-3/4-SOX2, enhancers, silencers, cAMP response elements, CAAT enhancer binding sequences, and insulators. Other response elements may also be included. A viral promoter may be replaced with a different promoter. The choice of the promoter will depend upon a number of factors, such as the proposed use of the HSV vector, treatment of the patient, disease state or condition, and ease of applying an inducer (for an inducible promoter). For treatment of cancer, generally when a promoter is replaced it will be with a cell-specific or tissue-specific or tumor-specific promoter. Tumor-specific, cell-specific and tissue-specific promoters are known in the art. Other gene elements may be modified as well. For example, the 5′ UTR of the viral gene may be replaced with an exogenous UTR.

### B. Specific Herpes Virus Constructs—VG2062

One preferred construct of the invention is provided in FIG. 1. Briefly, FIG. 1 diagrammatically depicts the overall structural organization of the double-stranded deoxyribonucleic acid (DNA) elements of VG2062. “CXCR4” means C-X-C Motif Chemokine Receptor 4; “CMV” means cytomegalovirus; “gB” means glycoprotein B; “ICP” means infected cell polypeptide; “IL” means interleukin; “R_{L}” means repeat long; “RNA” means ribonucleic acid; “miR” and “miRNA” means microRNA; “R_{S}” means repeat short; “UL” means unique long; “US” means unique short. The names “VG2062” and “VG203” refer to the same construct and may be used interchangeably.

VG2062 is a recombinant HSV-1 platform that utilizes both transcriptional and translational dual-regulation (“TTDR”—see FIG. 1) of key viral genes to limit virus replication to tumor cells and enhance tumor-specific virulence without compromising safety. In addition, VG2062 expresses a payload cassette composed of IL12, IL15 and IL15 receptor alpha subunit. The payload expression is under control of a CMV promoter for expression in cells that do not suppress CMV promoter function. Finally, a viral glycoprotein B (gB) in VG2062 was truncated to facilitate virus spread in the tumor by enhanced fusogenicity.

### C. Post-Transcriptional Regulation

In VG2062, ICP34.5 expression is post-transcriptionally regulated. Briefly, in wild-type HSV-1, there are 2 copies of the ICP34.5 gene. In VG2062, one copy of ICP34.5 has been deleted. For the remaining ICP34.5 gene, VG2062 inserts multiple copies of binding domains for miR124 and miR143 in the 3′UTR region to regulate its expression post-transcriptionally.

ICP34.5 is encoded by the HSV late gene g-34.5. It is well known for its function of suppressing anti-viral immunity of host cells, particularly neuronal cells, to cause neurotoxicity. To abolish the functions of ICP34.5 in neurons and other normal cells while retaining its activity in tumor cells for robust replication, instead of deleting the gene or using a specific promoter to control the expression of ICP34.5 to target specific tumors, VG2062 uses microRNAs as a post-transcriptional control to achieve differential expression of ICP34.5 in tumor cells. Briefly, miRNAs are ˜22 nucleotides, noncoding small RNAs coded by miRNA genes, which are transcribed by RNA polymerase II to produce primary miRNA (pri-miRNA). Mature single-stranded (ss) miRNA forms the miRNA-associated RNA-induced silencing complex (miRISC). miRNA in miRISC may influence gene expression by binding to the 3′-untranslated region (3′-UTR) in the target mRNA. This region consists of sequences recognized by miRNA. If the complementarity of the miRNA:mRNA complex is perfect, the mRNA is degraded by Ago2, a protein belonging to the Argonaute family. However, if the complementarity is not perfect, the translation of the target mRNA is not fully degraded, but is still suppressed.

miRNAs are expressed differentially in a tissue specific fashion. One of the examples is miR124. While the precursors of miR-124 from different species are different, the sequences of mature miR-124 in human, mice, rats are completely identical. MiR-124 is the most abundantly expressed miRNA in neuronal cells and is highly expressed in the immune cells and organs (Qin et al., 2016, miRNA-124 in immune system and immune disorders. Frontiers in Immunology, 7(OCT), 1-8). Another example of differential expression of miRNA is miR143 (Lagos-Quintana et al., 2002, Identification of tissue-specific MicroRNAs from mouse. Current Biology, 12(9), 735-739). MiR-143 is constitutively expressed in normal tissues but significantly downregulated in cancer cells (Michael et al., 2003, Reduced Accumulation of Specific MicroRNAs in Colorectal Neoplasia. Molecular Cancer Research, 1(12), 882-891).

The 3′ UTR region of ICP34.5 gene in VG2062 contains multiple copies of binding domains (also referred to as “miRNA target sequences”, “miRNA binding sequences” or “miRNA binding sites”) that are completely complementary to miR124 and miR143. Binding of miR124 and miR143 to the 3′UTR of ICP34.5 mRNA causes degradation of the mRNA; therefore the gene is post-transcriptionally downregulated in normal cells but not tumor cells. This design allows differential expression of ICP34.5 in tumor cells.

Within various embodiments, the miRNA target sequences can bind at least two different miRNAs (e.g., one or more of miR-124, miR-124*, and miR-143).). Within certain embodiments the miR target sequences include SEQ ID NO. 2 (a miR-124 binding sequence); SEQ ID NO. 3 (a miR-143 binding sequence); SEQ ID No. 9 (a miR-223 binding sequence); and/or SEQ ID NO. 10 (a miR-125b binding sequence). Other miRNA target sequences that can be utilized within other embodiments of the invention include, for example, mIR-122, miR-127, miR-128, miR-129, miR-129*, miR-132, mIR-133a, mIR133b, miR-135b, miR-136, miR-136*, miR-137, miR-139-5p, mIR-145, miR-154, miR-184, miR-188, miR-204, mIR216a, miR-299, miR-300-3p, miR-300-5p, miR-323, miR-329, miR-337, miR-335, miR-341, miR-369-3p, miR-369-5p, miR-376a, miR-376a*, miR-376b-3p, miR-376b-5p, miR-376c, miR-377, miR-379, miR-379*, miR-382, miR-382*, miR-409-5p, miR-410, miR-411, miR-431, miR-433, miR-434, miR-451, miR-466b, miR-485, miR-495, miR-539, miR-541, miR-543*, miR-551b, miR-758, and miR-873 as described in WO2020/113151, which is incorporated by reference in its entirety.

### D. Expression of ICP27 in VG2062 is Transcriptionally Controlled

HSV-1 viral replication depends on a cascade of expression of viral genes, with immediate early gene products (particularly ICP4 and ICP27) controlling subsequent expression of viral early genes and late genes that govern the lytic replication cycle of the virus. Deletion of ICP4 or ICP27 results in complete abrogation of viral replication and a significant reduction in viral gene expression, which makes ICP4 and ICP27 excellent targets for tumor specific regulation in oncolytic HSV.

While ICP4 is a major transcription factor regulating viral gene expression, ICP27 is a multi-functional protein that regulates transcription of many virus genes. ICP27 functions in all stages of mRNA biogenesis from transcription, RNA processing and export through to translation. ICP27 has also been implicated in nuclear protein quality control, cell cycle control, activation of stress signaling pathways and prevention of apoptosis.

In VG2062, the native promoter of ICP27 is replaced with a 279 bp promoter for human C-X-C motif chemokine receptor 4 (CXCR4) to enhance expression in urological tumors that typically have high levels of CXCR4.

### E. Payload Expression of VG2062 is Enhanced

VG2062 co-expresses IL12, IL15 and IL15 receptor alpha subunit to further stimulate an immunomodulatory response. Expression of IL12 promotes polarization of antigen exposed T cells towards an inflammatory and anti-tumor T_{H}1 phenotype, while IL-15 activates NK cells to further increase tumor killing and activation of antigen presenting cells. In addition to IL15 expression, VG2062 encodes IL15Rα to further enhance immune stimulation.

Transcription of IL-12, IL-15, and IL-15Rα and/or a PD-1, PD-L1 and/or PD-L2 antagonist can be driven by a single strong promoter (e.g., a viral promoter such as CMV, or, other strong constitutive promoters such as EF-1alpha or CAG, or, strong tumor-specific promoters such as CEA, CXCR4, PSA, ARR2PB, RAN, or telomerase) and the polypeptides are linked with 2A self-cleaving peptides (see SEQ ID NO. 7; see also Z. Liu et al., 2017, Systemic comparison of 2A peptides for cloning multi-genes in a polycistronic vector. Scientific Reports, 7(1), 1-9) that generate the 3 individual proteins through a mechanism of ribosomal skipping during translation.

### F. Truncated Glycoprotein B (GB)

HSV-1 membrane fusion is a crucial step during infection. It is dependent on four essential viral glycoproteins (gB, gD, gH, and gL), which mediate entry into host cells by merging the viral envelope with a host cell membrane. The core fusion protein is glycoprotein B (gB), a 904-residue glycosylated transmembrane protein encoded by the UL27 gene of HSV-1. Multiple types of mutations within the cytoplasmic domain of gB have yielded a hyperfusogenic phenotype, increasing cell-cell fusion (Chowdary & Heldwein, 2010, Syncytial Phenotype of C0Terminally Truncated Herpes Simplex Virus Type 1 gB Is Associated with Diminished Membrane Interactions. Journal of Virology, 84(10), 4923-4935). In one embodiment, gB may be modified by truncating C-terminal amino acids 877 to 904 from the full-length protein.

### G. Modified ICP47 Promoter

In the HSV genome, the promoter controlling expression of the US12 gene, which encodes UL47, is identical to the promoter controlling expression of the US1 gene, which is located approximately 13 k base pairs from the US12 gene. In addition, large regions of the native ICP47 promoter include repetitive sequences that may facilitate spurious homologous recombination events. Therefore, replacement of the native ICP47 promoter with a heterologous promoter is predicted to improve genomic stability of the virus.

In HSV, both ICP27 and ICP47 are encoded by immediate early genes, expressed very early after infection, and share many regulatory elements. Therefore, to reduce the risk of homologous recombination while maintaining a natural expression pattern, the native ICP47 promoter is replaced with the native ICP27 promoter in the VG2062 construct.

In some embodiments, the native ICP27 promoter includes the entire sequence of DNA located between the coding regions of UL53 (gK) and UL57 (ICP27). In one embodiment, the ICP27 promoter includes the 538 bp sequence set forth in SEQ ID NO. 1.

In other embodiments, the ICP27 promoter sequence may be 90%, 80%, 70%, 60%, or 50% identical to the ICP27 promoter sequence of any known human herpes virus type 1 strain or human herpes simplex virus type 2 strain, e.g., human herpes virus type 1 strain 17 (NCBI reference sequence NC_001806.2)

hVG2062 is an oncolytic virus product with ICP27 under control of the CXCR4 promoter, ICP34.5 under control of miRNA-124/143, and ICP47 under control of the ICP27 promoter. hVG2062 also incorporates a virus-expressed cytokine cassette encoding IL-12, IL-15/IL-15RA under the control of the CMV promoter. The expression control mechanisms in hVG2062 are designed to increase safety without sacrificing efficacy. Specific modifications to wild type—HSV-1 strain 17+ are set forth below in Table 1.

TABLE 1
Genetic Modification in VG2062 from wild type HSV-1, strain 17+

| | Modification | Modification | |
|---|---|---|---|
| Modification | Type | Location | Function |
| Deletion of both | Deletion | Terminal repeat | Wild type HSV-1 contains two |
| terminal repeat long | | long (TR_{L}) and | identical copies of TR_{L} and TR_{S}. |
| (TR_{L}) and short (TR_{S}) | | short (TR_{S}) | Removal of one copy may attenuate |
| containing ICP4, ICP0, | | | virulence while freeing up genomic |
| ICP34.5 and LAT genes | | | space for payload insertion. |
| Replacement of native | Replacement | ICP27 gene | To facilitate virus replication in |
| ICP27 promoter with | | promoter | CXCR4 positive tumor cells |
| CXCR4 promoter | | | |
| Insertion of binding | Insertion | ICP34.5 gene 3′- | To inhibit expression of ICP34.5 in |
| sites for miR143 and | | UTR | cells with high expression of miR143 |
| miR124 in the ICP34.5 | | | and/or miR124 |
| 3′-UTR | | | |
| Deletion of 84 bp in 3′ | Deletion | 3′ end of (gB) | To make HSV-1 gB protein truncated |
| end of glycoprotein B | | coding region | by 28 amino acids at its c-terminus |
| (gB) coding region | | | for enhanced fusogenicity |
| Insertion of IL-12, IL-15, | Insertion | Between UL3 and | To facilitate expression of IL-12, IL- |
| and IL-15Rα as an | | UL4 genes | 15, and IL-15Rα in all types of |
| expression cassette | | | infected cells that do not suppress |
| with CMV promoter | | | CMV promoter function |
| Replacement of native | Replacement | ICP47 gene | To reduce the risk of recombination |
| ICP47 promoter with | | promoter | events in the repetitive and non- |
| ICP27 promoter | | | unique ICP47 promoter region |

CEA = carcinoembryonic antigen; CXCR4 = C-X-C Motif Chemokine Receptor 4; gB = glycoprotein B; HSV-1 = herpes simplex virus-1; ICP0 = infected cell polypeptide 0; ICP27 = infected cell polypeptide 27; ICP47 = infected cell polypeptide 47; ICP34.5 = infected cell polypeptide 34.5; IL = interleukin; miR = microRNA; Rα = receptor alpha; TR_{L} = terminal repeat long; TR_{S} = terminal repeat short; UL = unique long; US = unique short; LAT = latency-associated transcript.

VG2062 is a conditionally replicating oncolytic HSV-1 virus. The genome of VG2062 deleted the terminal repeat long (TR_{L}) sequence of HSV-1 that contains one copy of ICP34.5, ICP0 and LAT and terminal repeat short (TR_{S}) sequence of HSV-1 that contains one copy of ICP4. The remaining copy of ICP34.5 has an insertion in its 3′UTR region containing multiple copies of binding domains for miRNA miR-124 and miR-143, which are highly expressed in neurons and normal tissues but not in tumor cells. The product is further modified by replacing the native viral promoter for the essential viral gene UL54 which encodes ICP27 (infected cell polypeptide 27) with a tumor-specific promotor from the tumor selective C-X-C Motif Chemokine Receptor 4 (CXCR4) gene. The product is further modified by replacing the native viral promoter driving expression of the immediate-early US12 gene which encodes ICP47 (infected cell polypeptide 47) with the native viral promoter which drives expression of the immediate-early UL54 gene which encodes ICP27 (infected cell polypeptide 27). VG2062 also expresses a potent immunomodulatory payload, consisting of IL-12, IL-15, and IL-15Rα, which is controlled by a cytomegalovirus (CMV promoter). Finally, VG2062 has a glycoprotein B (gB) truncation to enhance fusogenic activity, to facilitate virus spread within the tumor microenvironment.

### H. Therapeutic Compositions

Therapeutic compositions are provided that may be used to prevent, treat, or ameliorate the effects of a disease, such as, for example, cancer. More particularly, therapeutic compositions are provided comprising at least one oncolytic virus as described herein.

In certain embodiments, the compositions will further comprise a pharmaceutically acceptable carrier. The phrase “pharmaceutically acceptable carrier” is meant to encompass any carrier, diluent or excipient that does not interfere with the effectiveness of the biological activity of the oncolytic virus and that is not toxic to the subject to whom it is administered (see generally Remington: The Science and Practice of Pharmacy, Lippincott Williams & Wilkins; 21st ed. (May 1, 2005 and in The United States Pharmacopeia: The National Formulary (USP 40—NF 35 and Supplements).

In the case of an oncolytic virus as described herein, non-limiting examples of suitable pharmaceutical carriers include phosphate buffered saline solutions, water, emulsions (such as oil/water emulsions), various types of wetting agents, sterile solutions, and others. Additional pharmaceutically acceptable carriers include gels, bioabsorbable matrix materials, implantation elements containing the oncolytic virus, or any other suitable vehicle, delivery or dispensing means or material(s). Such carriers can be formulated by conventional methods and can be administered to the subject at an effective dose. Additional pharmaceutically acceptable excipients include, but are not limited to, water, saline, polyethylene glycol, hyaluronic acid and ethanol. Pharmaceutically acceptable salts can also be included therein, e.g., mineral acid salts (such as hydrochlorides, hydrobromides, phosphates, sulfates, and the like) and the salts of organic acids (such as acetates, propionates, malonates, benzoates, and the like). Such pharmaceutically acceptable (pharmaceutical-grade) carriers, diluents and excipients that may be used to deliver the oHSV to a cancer cell will preferably not induce an immune response in the individual (subject) receiving the composition (and will preferably be administered without undue toxicity).

The compositions provided herein can be provided at a variety of concentrations. For example, dosages of oncolytic virus can be provided which ranges from about 10⁶ to about 109 pfu. Within further embodiments, the dosage can range from about 10⁶ to about 10⁸ pfu/ml, with up to 4 mls being injected into a patient with large lesions (e.g., >5 cm) and smaller amounts (e.g., up to 0.1 mls) in patients with small lesions (e.g., <0.5 cm) every 2-3 weeks, of treatment.

Within certain embodiments of the invention, lower dosages than standard may be utilized. Hence, within certain embodiments less than about 10⁶ pfu/ml (with up to 4 mls being injected into a patient every 2-3 weeks) can be administered to a patient.

The compositions may be stored at a temperature conducive to stable shelf-life and includes room temperature (about 20° C.), 4° C., −20° C., −80° C., and in liquid N2. Because compositions intended for use in vivo generally do not have preservatives, storage will generally be at colder temperatures. Compositions may be stored dry (e.g., lyophilized) or in liquid form.

### I. Administration

In addition to the compositions described herein, various methods of using such compositions to treat or ameliorate cancer are provided, comprising the step of administering an effective dose or amount of oHSV as described herein to a subject.

The terms “effective dose” and “effective amount” refers to amounts of the oncolytic virus that is sufficient to effect treatment of a targeted cancer, e.g., amounts that are effective to reduce a targeted tumor size or load, or otherwise hinder the growth rate of targeted tumor cells. More particularly, such terms refer to amounts of oncolytic virus that is effective, at the necessary dosages and periods of treatment, to achieve a desired result. For example, in the context of treating a cancer, an effective amount of the compositions described herein is an amount that induces remission, reduces tumor burden, and/or prevents tumor spread or growth of the cancer. Effective amounts may vary according to factors such as the subject's disease state, age, gender, and weight, as well as the pharmaceutical formulation, the route of administration, and the like, but can nevertheless be routinely determined by one skilled in the art.

The therapeutic compositions are administered to a subject diagnosed with cancer or is suspected of having a cancer. Subjects may be human or non-human animals.

The compositions are used to treat cancer. The terms “treat” or “treating” or “treatment,” as used herein, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. The terms “treating” and “treatment” can also mean prolonging survival as compared to expected survival if not receiving treatment.

Representative forms of cancer include carcinomas, leukemias, lymphomas, myelomas and sarcomas. Representative forms of leukemias include acute myeloid leukemia (AML) and representative forms of lymphoma include B cell lymphomas. Further examples include, but are not limited to urological cancers, cancer of the bile duct, brain (e.g., glioblastoma), breast, cervix, colorectal, CNS (e.g., acoustic neuroma, astrocytoma, craniopharyogioma, ependymoma, glioblastoma, hemangioblastoma, medulloblastoma, menangioma, neuroblastoma, oligodendroglioma, pinealoma and retinoblastoma), endometrial lining, hematopoietic cells (e.g., leukemia's and lymphomas), kidney, bladder, larynx, lung, liver, oral cavity, ovaries, pancreas, prostate, skin (e.g., melanoma and squamous cell carcinoma), GI (e.g., esophagus, stomach, and colon) and thyroid. Cancers can comprise solid tumors (e.g., sarcomas such as fibrosarcoma, myxosarcoma, liposarcoma, chondrosarcoma and osteogenic sarcoma), be diffuse (e.g., leukemia's), or some combination of these (e.g., a metastatic cancer having both solid tumors and disseminated or diffuse cancer cells). Cancers can also be resistant to conventional treatment (e.g. conventional chemotherapy and/or radiation therapy). Benign tumors and other conditions of unwanted cell proliferation may also be treated.

Particularly preferred cancers to be treated include those with high levels of CXCR4 expression. Representative examples include urological tumors, lung tumors, breast and prostate tumors, glioblastomas, tumors of the gastro-intestinal tract (and associated organs) e.g., esophagus, cholangiocarcinoma, anal, stomach, bladder, intestine, pancreatic, colon and liver, and all surface injectable tumors (e.g., melanomas).

The recombinant herpes simplex viruses described herein may be given by a route that is e.g. oral, topical, parenteral, systemic, intravenous, intramuscular, intraocular, intrathecal, intratumoral, subcutaneous, or transdermal. Within certain embodiments the oncolytic virus may be delivered by a cannula, by a catheter, or by direct injection. The site of administration may be directly into the tumor, adjacent to the tumor, or at a site distant from the tumor. The route of administration will often depend on the type of cancer being targeted.

The optimal or appropriate dosage regimen of the oncolytic virus is readily determinable within the skill of the art, by the attending physician based on patient data, patient observations, and various clinical factors, including for example a subject's size, body surface area, age, gender, and the particular oncolytic virus being administered, the time and route of administration, the type of cancer being treated, the general health of the patient, and other drug therapies to which the patient is being subjected.

Recombinant herpes simplex viruses described herein may be formulated as medicaments and pharmaceutical compositions for clinical use and may be combined with a pharmaceutically acceptable carrier, diluent, excipient or adjuvant. The formulation will depend, at least in part, on the route of administration. Suitable formulations may comprise the virus and inhibitor in a sterile medium. The formulations can be fluid, gel, paste or solid forms. Formulations may be provided to a subject or medical professional.

A therapeutically effective amount is preferably administered. This is an amount that is sufficient to show benefit to the subject. The actual amount administered, and the time-course of administration will depend at least in part on the nature of the cancer, the condition of the subject, site of delivery, and other factors.

Within yet other embodiments of the invention the oncolytic viruses provided herein may be administered along with (e.g., prior to, at the same time or subsequently) a therapeutic agent that blocks, binds to, or otherwise inhibits CTLA-4, PD-1, or a ligand thereof. Briefly, CTLA-4 (or “cytotoxic T-lymphocyte-associated protein 4”, and also known as CD152) and PD-1 (or “Programmed cell death protein 1”, and also known as CD279) are proteins on the surface of T and B cells that have a role in down regulating the immune system, thereby suppressing T cell inflammatory activity and preventing or decreasing the ability of the immune system to kill tumor cells.

PD-1 has two ligands: PD-L1 and PD-L2. Representative examples of PD-1, PD-L1 and PD-L2 antagonists include peptides and antibodies that block, bind to or otherwise inhibits the binding of PD-1 or it's ligands in a statistically significant manner, thereby inhibiting or diminishing their activity. Representative examples of PD-L1 antagonists include: Atezolizumab (TECENTRIQ—a humanized anti-PD-L1 antibody); Avelumab (BAVENCIO—a fully human anti-PD-L1 antibody); Durvalumab (IMFINZI—a human anti-PD-L1 antibody); Durvalumab (IMFINZI—a fully human igG1 antibody); KN035; Cosibelimab (CK-301 by Checkpoint Therapeutics; AUNP12 (a peptide PD-1/PD-L1 inhibitor by Aurigene and Laboratoires Pierre Fabre); CA-170; and BMC-986189 (a macrocyclic peptide).

Representative examples of PD-1 antagonists include the following representative anti-PD-1 antibodies: Nivolumab (OPDIVO—a human anti-PD-1 antibody); Pembrolizumab (KEYTRUDA—a humanized anti-PD-1 antibody); Cemiplimab (LIBTAYO—an anti-PD-1 antibody); Dostarlimab (JEMPERLI—an anti-PD-1 antibody; Vopratelimab (JTX-4014) by Jounce Therapeutics; Spartalizumab (PDR001) by Novartis; Camrelizumab (SHR1210)—an anti-PD-1 monoclonal antibody by Jiangsu HengRui Medicine Co., Ltd.; Sintilimab (161308), a human anti-PD-1 antibody developed by Innovent and Eli Lilly; Tislelizumab (BGB-A317)—a humanized IgG4 anti-PD-1 monoclonal antibody; Toripalimab (JS 001) a humanized IgG4 monoclonal antibody against PD-1; INCMGA00012 (MGA012) a humanized IgG4 monoclonal antibody developed by Incyte and MacroGenics; AMP-224 by AstraZeneca/MedImmune and GlaxoSmithKline; and AMP-514 (MEDI0680) by AstraZeneca.

Representative patents and patent applications describing PD-1, PD-L1 and PD-L2 antagonists and methods for assessing their activity include, for example: U.S. Pat. Nos. 7,595,048, 7,943,743, 8,952,136, 8,217,149, 8,609,089, 8,735,553, 8,779,105, 8,779,108, 8,993,731, and 9,815,897; U.S. Publication Nos. 2010/0203056, 2010/0266617, 2011/0229461, 2013/0017199, 2014/341917, 2015/0203579, 2016/0311903, 2016/0376367, 2016/0311903, 2017/0044259 and 2018/0346569; and PCT Publication No. WO2012145493; all of which are incorporated by reference in their entirety.

CTLA-4 also has two primary ligands: CD80 and CD86 on the surface of T cells. Representative examples of CTLA-4 antagonists include peptides and antibodies that block, bind to or otherwise inhibits the binding of CTLA-4 or it's ligands in a statistically significant manner, thereby inhibiting or diminishing their activity. Representative examples of CTLA-4 antagonists include: Ipilimumab (Yervoy—a fully human anti-CTLA-4 monoclonal antibody, IgG1 isotype); and Tremelimumab (Imjudo—a fully human anti-CTLA-4 antibody, IgG2 isotype). Representative examples of patents that relate to anti-CTLA-4 antibodies and variants thereof include U.S. Pat. Nos. 5,811,097, 5,855,887, 5,977,318, 6,051,227, 6,207,156, 6,682,736, 6,984,720, 7,109,003, 7,132,281 and 10,196,445, and PCT Publication Nos. WO 2001/014424, WO 2004/035607, 2005/0201994, all of which are incorporate by reference in their entirety.

According to certain embodiments, treatment of a subject using the oncolytic virus and a CTLA-4, PD-1, PD-L1 or PD-L2 antagonist as described herein may be combined with additional types of therapy, such as surgical resection, administration of a different oncolytic virus, radiotherapy, administration of a checkpoint inhibitor, chemotherapy using, e.g., a chemotherapeutic agent such as etoposide, ifosfamide, adriamycin, vincristine, doxycycline, and others.

### EXAMPLES

Overview: All viral mutagenesis can be performed in Escherichia coli using standard lambda Red-mediated recombineering techniques implemented on the viral genome cloned into a bacterial artificial chromosome (BAC) (see generally: Tischer B K, Smith G A, Osterrieder N. Methods Mol Biol. 2010; 634:421-30. doi: 10.1007/978-1-60761-652-8_30. PMID: 20677001; Tischer B K, von Einem J, Kaufer B, and Osterrieder N., BioTechniques 40:191-197, February 2006 (including the Supplementary Material, doi: 10.2144/000112096; and Tischer B K, Smith, G A and Osterrieder N. Chapter 30, Jeff Braman (ed.), In Vitro Mutagenesis Protocols: Third Edition, Methods in Molecular Biology, vol. 634, doi: 10.1007/978-1-60761-652-8_30, Springer Science+Business Media, LLC 2010).

BAC recombineering requires the presence of exogenous BAC DNA within the viral genome to facilitate mutagenesis in E. coli. The BAC sequence is most commonly inserted either between viral genes such as the HSV genes US1/US2, UL3/UL4 and/or UL50/UL51, or, into the thymidine kinase (TK) gene, which can disrupt expression of native TK. TK-deficient viral vectors may include an expression cassette for a copy of the native viral thymidine kinase (TK) gene under the control of a constitutive promoter inserted into a non-coding region of the viral genome. Alternatively, TK function may be restored by removing the exogenous BAC sequences via homologous recombination to reconstitute the native TK gene sequence. Presence of a functional TK gene enhances virus safety by rendering the virus sensitive to common treatment with guanosine analogues, such as ganciclovir and acyclovir.

### Example 1

### Development of TTDR Virus Platform VG203

As shown in FIG. 1, transcriptional regulation is accomplished by utilizing the tumor-specific CXCR4 promoter to control expression of the essential HSV-1 transactivator protein ICP27. Translational regulation employs multiple tandem copies of microRNA binding sites inserted into the 3′-UTR of the key HSV-1 neurovirulence factor ICP34.5 which promote the binding of microRNAs that are both abundant in normal cells and downregulated in tumor cells. Attachment of said microRNAs leads to reduced translation and increased degradation of the ICP34.5 mRNA transcript in normal cells, while allowing ICP34.5 production to proceed at near wild-type levels in tumor cells. Additionally, glycoprotein B was truncated to increase fusogenicity and a CMV promoter-driven cytokine expression cassette encoding IL-12, IL-15, and IL-15 alpha receptor was inserted between HSV-1 genes UL3 and UL4. The native promoter driving expression of ICP47 was also replaced by the HSV-1 ICP27 promoter to reduce the risk of recombination events in the repetitive and non-unique ICP47 promoter region. Wild-type HSV-1 contains two identical copies of RL (containing ICP0 and ICP34.5) and RS (containing ICP4) that facilitate recombination events during viral replication, yielding four roughly equimolar isomers of the HSV-1 genome where the orientation of the US and UL regions is inverted (see generally, Slobedman B, Zhang X, Simmons A. Herpes simplex virus genome isomerization: origins of adjacent long segments in concatemeric viral DNA. J Virol. 1999 January; 73(1):810-3. doi: 10.1128/JVI.73.1.810-813.1999. PMID: 9847394; PMCID: PMC103895). Genome linearization occurs at the RL-RS junction, and the separated RL and RS flanking the genome are typically called the TRL (terminal repeat long) and TRS (terminal repeat short) while the unseparated internal set of RL and RS are known as IRL (internal repeat long) and IRS (internal repeat short). We removed one copy of both RL and RS to reduce the probability of internal recombination and to lock the viral genome into a single stable configuration, while simultaneously attenuating virulence and freeing up genomic space for payload insertion.

CXCR4=C-X-C Motif Chemokine Receptor 4; gB=glycoprotein B; HSV-1=herpes simplex virus-1; ICP0=infected cell polypeptide 0; ICP27=infected cell polypeptide 27; ICP47=infected cell polypeptide 47; ICP34.5=infected cell polypeptide 34.5; IL=interleukin; miR=microRNA; TRL=terminal repeat long; TRS=terminal repeat short; UL=unique long; US=unique short. VG2062 is a historical name for VG203 and may be used interchangeably.

### Example 2

### MicroRNA-Mediated Control of Viral Gene Expression

Objective To investigate the ability of microRNAs to control expression of viral genes.

Procedure: 293T cells were transfected with precursors for either miR-124 and miR-143 or miR-223 and miR-125b. Transfection with scrambled miR served as the negative control. 24 hours post-transfection, cells were superinfected with VG17 (wild-type), VG161, mVG2031-TK, VG2062-TK, or two different clones of VG21224-TK. VG17 and VG161 served as negative controls as they do not incorporate exogenous miR binding sites, while mVG2031-TK is a murine version of VG2062 containing mouse IL-12 in place of human IL-12. VG21224-TK is an alternative name for VG21224 (see PCT/CN2023/073525), and VG2062-tk is an alternative name for VG2062. The difference between VG21224 and VG2062 lies in the presence of 5 tandem copies of binding sites for miR-223 and 5 tandem copies of binding sites for miR-125b located in the 3-′UTR of ICP27 in VG21224. RNA was isolated 6 hours post infection and RT-qPCR was performed to measure levels of ICP27 and ICP34.5 transcripts.

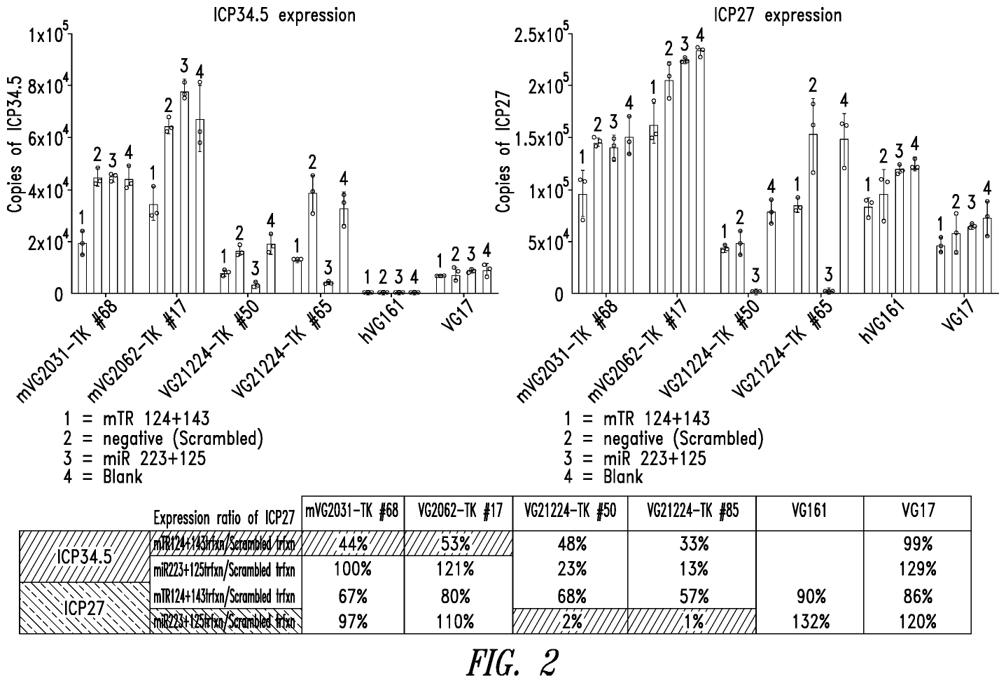

Results: As shown in FIG. 2, the presence of miR-124 and miR-143 resulted in roughly a 50% reduction in ICP34.5 transcripts for all viruses that incorporate miR-124 and miR-143 binding sites in the 3′-UTR of ICP34.5 (mVG2031-TK, VG2062-TK, and VG21224-TK). Regulation of ICP27 using miR-223 and miR-125b was even more effective, resulting in nearly complete reduction of ICP27 expression in both tested clones of VG21224-TK. Interestingly, miR-223 and miR-125b were even more effective at downregulating ICP34.5 transcripts than miR-124 and miR-143 in VG21224-TK, despite the lack of miR-223 and miR-125b binding sites in the 3′-UTR of ICP34.5, likely due to effective regulation of ICP27 via miR-223 and miR-125b in VG21224-TK. ICP27 is a key transactivator of other viral genes including ICP34.5, explaining why downregulation of ICP27 expression would also result in reduced expression of other viral genes, ultimately leading to reduced replication efficiency in non-tumor cells that express high levels of miR-223 and miR-125b.

Conclusion: microRNAs are capable of effectively controlling expression of viral genes in this experimental system.

### Example 3

### Comparison of Cell-to-Cell Fusion Phenotypes

Objective: To evaluate the ability of different recombinant HSVs to cause cell-to-cell fusion in vitro.

Procedure: Vero cell monolayers were seeded in 96-well plates and infected with VG21224 (VG202), VG2062 (VG203), and VG2063. Viral plaques were imaged 3 days post infection at 10× magnification. VG2063 differs from VG2062 in retaining the native ICP47 promoter, while both VG2062 and VG21224 have the native ICP47 promoter replaced with the HSV-1 ICP27 promoter.

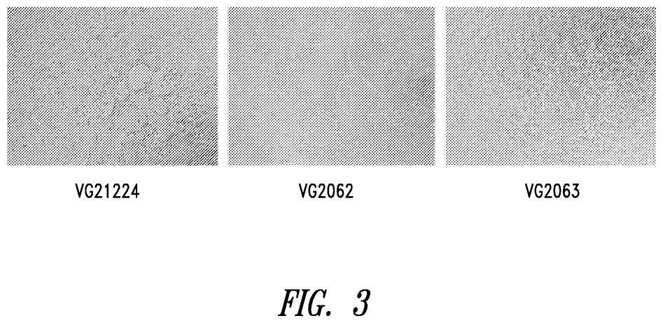

Results: As shown in FIG. 3, VG2063 exhibits only mild fusogenicity. However, we observed a significant and unexpected increase in cell-to-cell fusion when the native ICP47 promoter was replaced with the ICP27 promoter as evidenced by the presence of numerous multinucleated giant cells (syncytia) in Vero cells infected with VG21224 and VG2062. This result is significant because ICP47 has no known role in cell-to-cell fusion and no syncytial mutations in ICP47 have ever been documented.

Conclusion: Replacing the native ICP47 promoter with the native ICP27 promoter yields an unexpected increase in syncytia formation.

### Example 4

### HSV Genome Stability Over Multiple Passages In Vitro

Objective: To evaluate the stability of the UL-US junction after deleting one copy of RL and one copy of RS located between the genes UL1 and US12 (ICP47). We hypothesized that replacing the native ICP47 promoter should have a positive effect on HSV genomic stability because the promoter driving the US12 gene which encodes ICP47 is identical to the promoter of the US1 gene, which is located approximately 13 kbp apart from US12 on the opposite side of the US region. Moreover, large portions of the native ICP47 promoter are comprised of repetitive sequences which may facilitate spurious homologous recombination events.

Procedure: VG2062 virus containing a deletion of one copy of RL and one copy of RS located between the genes UL1 and US12 and with the US12 (ICP47) promoter replaced with the ICP27 promoter was passaged multiple times on Vero cells. Stability of the DNA region located between the genes UL1 and US12 was determined by using primers that anneal to the UL1 and US12 coding regions and amplifying the region located between the genes UL1 and US12 by PCR. The PCR result was compared to that of a control virus that also contains a deletion of one copy of RL and one copy of RS located between the genes UL1 and US12 but retains the native US12 (ICP47) promoter. The control virus was also subjected to multiple passages on Vero cells.

Results: We observed no change in the electrophoretic mobility of the PCR-amplified DNA fragment corresponding to the UL-US junction in VG2062 even after multiple passages in vitro. The PCR-amplified DNA fragments were also sequenced and no changes in the DNA sequence were observed between passages. This result lies in contrast to the control virus, where the UL-US junction could not be amplified by PCR after several passages in vitro.

Conclusion: Replacement of the native ICP47 promoter with the ICP27 promoter increases the genomic stability of the HSV UL-US junction after deleting one copy of RL and one copy of RS from said UL-US junction, where the UL-US junction is flanked by UL1 and US12 (ICP47).

### Example 5

### Analysis of HSV-1 ICP27 Gene Regulation by CXCR4 Promoter in VG2062

Objective: C-X-C motif chemokine receptor 4 (CXCR4) is involved in the growth of tumors and their metastasis throughout the body. As part of the Transcription and Translation Dual Regulated (TTDR) strategy employed in VG2062, the native promoter of the HSV-1 gene ICP27 was replaced with the tumor-specific CXCR4 promoter. This study evaluated 9 urinary carcinoma cell lines by detecting ICP27 gene DNA to correlate VG2062 replication with CXCR4 expression in each cell line, and by quantifying VG2062 growth in infected cells by plaque assay.

Procedure: Cell lines T24, DU145, UM-UC-3, HT-1376, 786-0, LNCap, J82, PC3, and 22Rv1 were grown in T182 flasks with appropriate growth medium. At day 0, cells were trypsinized and seeded into 24-well plates. At 0 h of day 1, cells were infected for 2 hours with VG2062 at MOI=0.001 (done in triplicate for each sample). Samples were harvested at 2 hours, 24 hours, 48 hours, 72 hours, and 96 hours. Cell lysates were used for DNA/mRNA extraction and the supernatants were used for virus titration. DNA and mRNA was isolated following the protocol provided by the kit vendor, and purified DNA and mRNA was eluted in 30 μl nuclease-free water. The mRNAs (200 ng) were reverse transcribed and the cDNAs were collected in 20 μl ddH2O and subsequently diluted 1:10 with 180 μl ddH2O. The cDNAs were subjected to qPCR to measure CXCR4 gene expression relative to expression of the housekeeper gene GAPDH, while the DNA was also subjected to qPCR to measure ICP27 gene copy numbers relative to GAPDH.

For plaque assay, infected cell supernatants were subjected to serial 10-fold dilutions with serum free media, and 500 μl of the diluted samples were used to infect Vero cells in 12-well plates. Infected Vero cells were overlaid with 500 μl of gelatinous medium (MEM+1.5% methylcellulose+1% FBS) after 2 hours of incubation. Plates were then kept in a CO2 incubator for 3 days, at which point the gelatinous medium was aspirated and discarded. The cells were fixed by the addition of 500 μl 4% glutaraldehyde for 2 hours. 500 μl 0.5% crystal violet solution was added to each well, and the plates were washed and air dried. The number of plaques was counted and recorded from each well.

Results: RNA samples isolated from VG2062-infected cells were used to measure mRNA expression levels of the CXCR4 gene and cellular GAPDH, while DNA samples isolated from VG2062-infected cells were used to measure the DNA levels of viral ICP27 and cellular GAPDH. Samples were collected from 3 replicate wells in 24-well plates. The average cycle threshold (Ct) values were determined from 2 replicate wells from each sample. The Ct Value of CXCR4 minus the Ct Value of GAPDH was used to obtain ΔCt (CXCR4-GAPDH, 0 h). The Ct Value of ICP27 minus the Ct Value of GAPDH was used to obtain ΔCt (ICP27-GAPDH) for each time point, and the 72 hour time point of ΔCt (ICP27-GAPDH) minus the 2 hour timepoint was used to calculate ΔCt (ICP27-GAPDH, 72 h-2 h), which corresponds to the incremental change in viral DNA copy number.

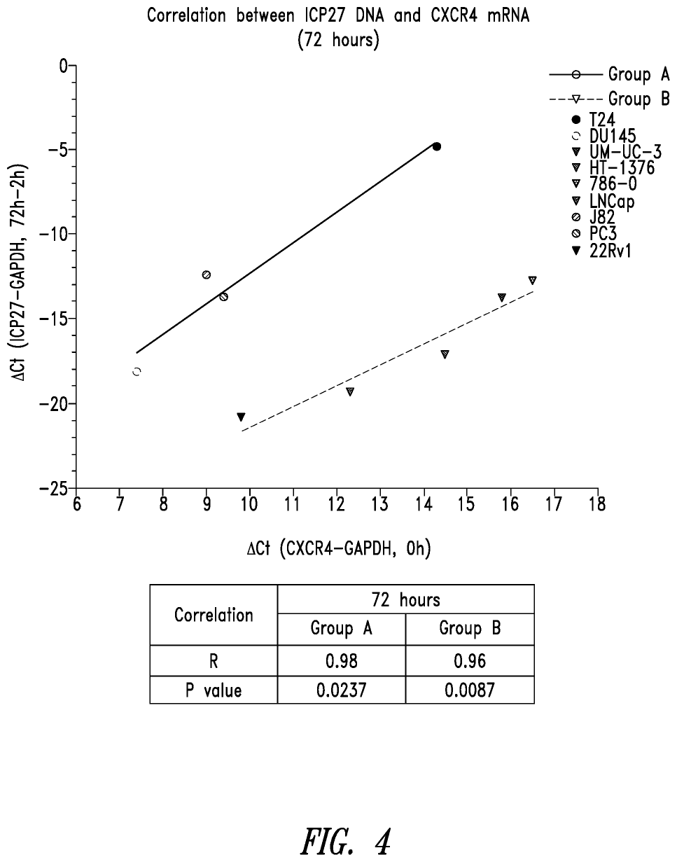

The data were statistically analyzed after averaging 3 parallel data sets from each cell line. The respective correlation (R) and P values between ICP27 DNA and CXCR4 mRNA were determined using GraphPad Prism software 8.0. The 9 cell lines were divided into 2 groups based on linear fitting as depicted in FIG. 4. Group A consisted of the T24, DU145, J82 and PC3 cell lines. At 72 h post infection, viral ICP27 DNA levels positively correlated with the background levels of CXCR4 mRNA in each cell line (R=0.98, P=0.0237). Group B consisted of the UM-UC-3, HT-1376, 786-0, LNCap, and 22Rv1 cell lines. At 72 h post infection, viral ICP27 DNA levels also positively correlated with the background levels of CXCR4 mRNA in each cell line (R=0.96, P=0.0087).

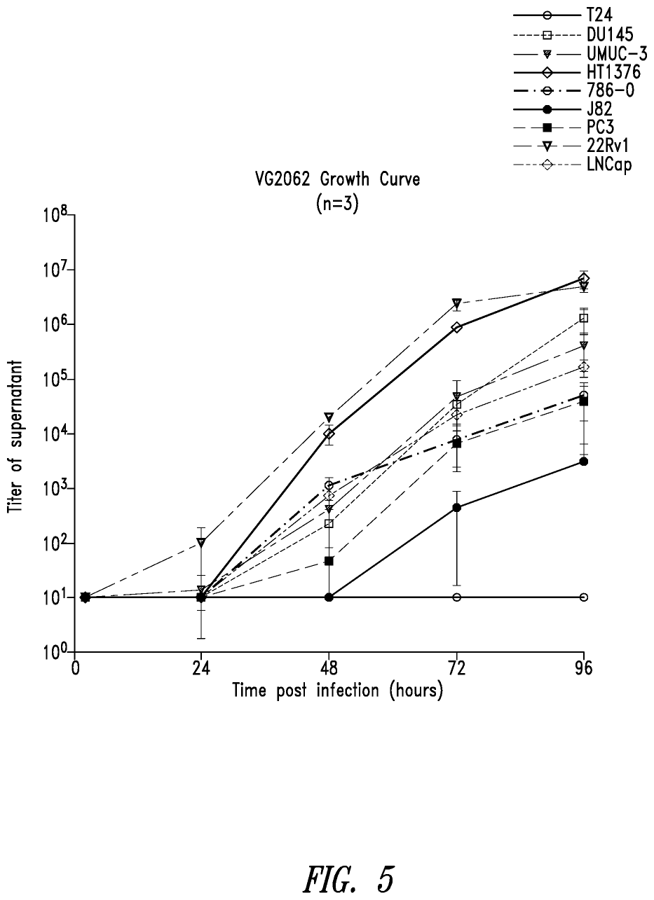

The replication kinetics of VG2062 in the 9 tested cell lines at 5 time points were quantified by plaque assay (FIG. 5). VG2062 propagated poorly on T24 cells (below the limit of detection of plaque assay: 10 PFU/mL), which was consistent with the results observed in FIG. 4 (high CT numbers in qPCR reactions for ICP27 and CXCR4 from T24 cell samples). VG2062 was able to propagate on the remaining 8 tumor cell lines.

Overall, the observed robust correlation between VG2062 viral DNA production and CXCR4 expression levels in infected cells strongly supports our Transcription and Translation Dual Regulated (TTDR) strategy for recombinant OV construction.

### Example 6

### Cytotoxicity of VG2062 in Different Urological Tumor Cell Lines

Objective: To compare cytotoxicity of the TTDR virus VG2062 with the non-TTDR virus VG161 in a panel of 10 different urological tumor cell lines.

Procedure: T24, DU145, ACHN, UMUC-3, HT1376, 786-0, LNCap, J82, PC3, and Caki-1 cells were seeded into 96-well plates and treated with different MOI of either VG161 or VG2062. OD450 nm was read to evaluate cell viability at 24 h, 48 h, and 96 h after infection.

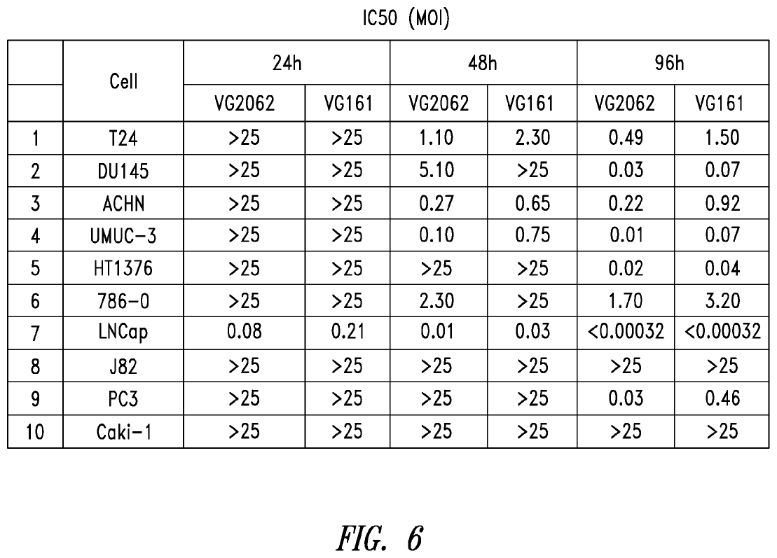

Results: The results are provided in FIG. 6. LNCap cells were highly susceptible to HSV-mediated cytotoxicity by both viruses at all tested time points, while J82 cells and Caki-1 cells were resistant to HSV-mediated cell killing even at the 96 h time point. In all cell lines and time points with measurable cytotoxicity (IC50 MOI<25), the VG2062 virus consistently exhibited increased cell killing when compared to VG161, with VG2062 IC50 MOI values ranging between 2-fold and more than 15-fold lower than for VG161.

### Example 7

### Quantification of Cytokine Payload Expression

Objective: To measure human IL-12 and human IL-15 payload secretion from VG161 and VG2062 virally-infected urological tumor cell lines.

Procedure: T24, DU145, ACHN, UMUC-3, HT1376, 786-0, LNCap, J82, PC3, and Caki-1 cells were seeded in 12-well plates at 37′C overnight and subsequently infected with VG161 (non-TTDR virus) or VG2062 (TTDR virus), respectively. Cell supernatants were harvested and quantified for human IL 12p70 and human IL-15/IL-15Rα by ELISA.

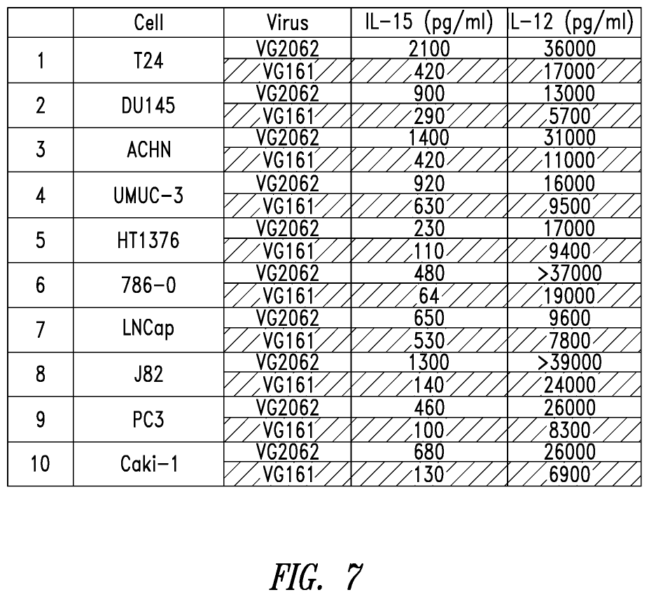

Results: Detectable levels of IL-12 and IL15 were observed in all tested viruses and cell lines, but the IL-12 and IL-15 levels in VG2062-infected cells were universally higher compared to IL-12 and IL-15 from cells infected with VG161 (FIG. 7).

### Example 8

### Evaluation of VG2062 Efficacy in the A549 Human Lung Cancer Xenograft Model

Objective: To evaluate the anti-tumor efficacy of VG2062 administered intratumorally (i.t.) in the A549 human lung cancer xenograft model in female BALB/c nude mice.

Procedure: 12 female BALB/c nude mice were subcutaneously implanted with A549 cells. Mice were randomized into 2 groups, with 8 mice in the treatment group and 4 mice in the control group. The day of randomization was defined as day 0. Group 2 was administered DPBS+7.5% glycerin (vehicle control). Group 1 received 3 intratumoral doses of VG2062 (2.4×10⁷ PFU/mouse/dose, 0.1 mL/animal) at days 1, 2, and 3. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

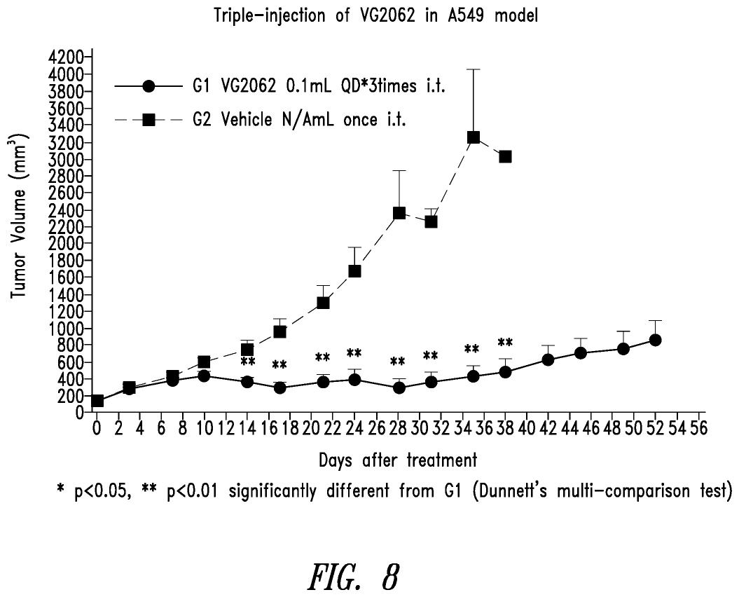

Results: There was no statistical difference in tumor volume at the time of randomization (Day 0). During the experiment, the tumor volumes of the vehicle control group (Group 2) mice continued to increase, while tumor volumes in mice treated with 3 doses of 2.4×10⁷ PFU/mouse/dose of VG2062 remained relatively stable (FIG. 8). Compared to the vehicle control group, the virus-treated group showed statistically significant inhibition of tumor growth (P<0.05) at 14 days post injection and at every timepoint thereafter. VG2062 was well tolerated by the mice during this study, and these data indicate that VG2062 may have a significant anti-tumor effect in lung cancer cells such as A549.

### Example 9

### Dose-Escalation Study in Prostate Cancer PC3-Bearing Mice

Objective: To evaluate the anti-tumor activity of different dosages of VG2062 on mice xenografted with the PC3 cell line (human prostate cancer).

Procedure: 40 SPF-grade male nude mice (4-5 weeks old) were subcutaneously implanted with 5×10⁶ PC3 cells/mouse. The inoculation site was the right side of the mouse's back near the armpit. Mice were randomized into 5 groups, with 8 mice in each group. The day of group dosing was defined as day 0. Group 5 was administered DPBS+7.5% glycerin (vehicle control). Groups 1 to 4 were test groups receiving a single dose of VG2062 (0.1 mL/animal) at different dose levels via intratumoral injection. Group 1 received a dose of 2.4×10⁷ PFU/mouse, group 2 received a dose of 2.4×10⁶ PFU/mouse, group 3 received a dose of 2.4×10⁵ PFU/mouse, and group 4 received a dose of 2.4×10⁴ PFU/mouse. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

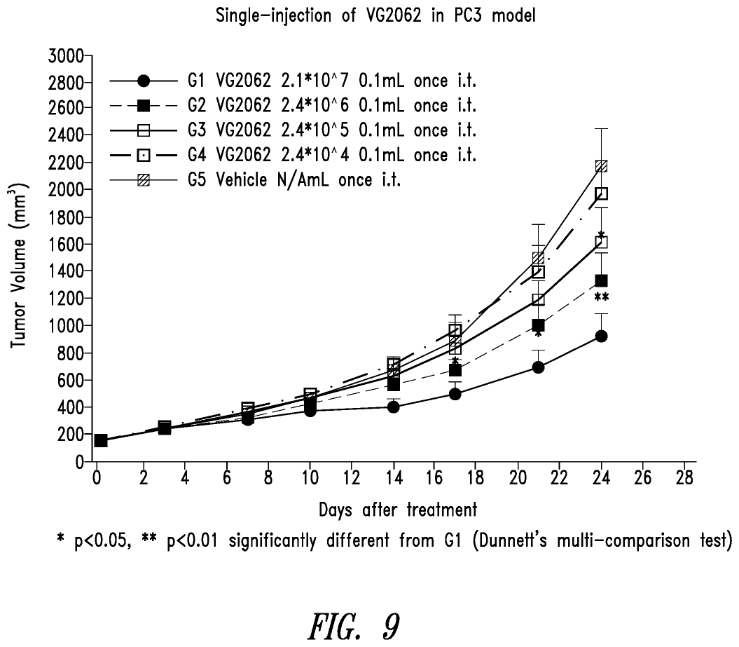

Results: There was no statistical difference in tumor volume at the time of randomization (Day 0). During the experiment, the tumor volumes of the vehicle control group (Group 5) mice continued to increase—the tumor volume on day 0 was 149.85±2.18 mm³, which grew to 3151.38±325.33 mm³ on day 28 (FIG. 9). In Group 1 (2.4×10⁷ PFU/mouse), tumor volume on day 28 was 1297.80±196.40 mm³. In Group 2 (2.4×10⁶ PFU/mouse), tumor volume on day 28 was 1913.91±246.23 mm³. In Group 3 (2.4×10⁵ PFU/mouse), tumor volume on day 28 was 2175.27±4292.09 mm³. In Group 4 (2.4×10⁴ PFU/mouse), tumor volume on day 28 was 3032.35±422.25 mm³. Compared to the vehicle control group, groups 1 and 2 both showed statistically significant inhibition of tumor growth (P<0.05). VG2062 was well tolerated by the mice during this study, and these data indicate that VG2062 may have a significant anti-tumor effect in prostate cancer cells such as PC3 starting at a dosage of 2.4×10⁶ PFU/mouse.

### Example 10

### Dose-Escalation Study in Prostate Cancer DU145-Bearing Mice

Objective: To evaluate the anti-tumor activity of different dosages of VG2062 on mice xenografted with the DU145 cell line (human prostate cancer).

Procedure: 40 SPF-grade nude mice were subcutaneously implanted with DU145 cells. Mice were randomized into 5 groups, with 8 mice in each group. The day of group dosing was defined as day 0. Group 5 was administered DPBS+7.5% glycerin (vehicle control). Groups 1 to 4 were test groups receiving a single dose of VG2062 (0.1 mL/animal) at different dose levels via intratumoral injection. Group 1 received a dose of 2.4×10⁷ PFU/mouse, group 2 received a dose of 2.4×10⁶ PFU/mouse, group 3 received a dose of 2.4×10⁵ PFU/mouse, and group 4 received a dose of 2.4×10⁴ PFU/mouse. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

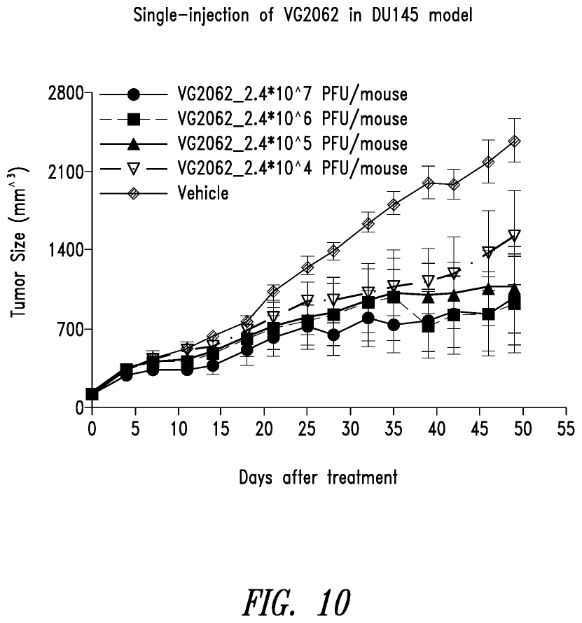

Results: There was no statistical difference in tumor volume at the time of randomization (Day 0). During the experiment, the tumor volumes of the vehicle control group (Group 5) mice increased at a higher rate than tumor volumes of mice treated with VG2062 (FIG. 10). Compared to the vehicle control group, groups 1, 2, and 3 all showed statistically significant inhibition of tumor growth (P<0.05). VG2062 was well tolerated by the mice during this study, and these data indicate that VG2062 may have a significant anti-tumor effect in prostate cancer cells such as DU145 starting at a dosage of 2.4×10⁵ PFU/mouse.

### Example 11

### Treatment of Bladder Cancer UM-UC-3-Bearing Nude Mice

Objective: To evaluate the efficacy of VG2062 in a xenograft model of UM-UC-3 human bladder cancer cells.

Procedure: 24 SPF-grade female nude mice were subcutaneously implanted with 5×10⁶ UM-UC-3 cells/mouse (5×10⁷ cells/mL, 0.1 mL/mouse) and randomized into 3 groups, with 8 mice in each group. Group 1 was administered DPBS+7.5% glycerin (vehicle control). Groups 2 and 3 were high and low dose test groups receiving a single dose of VG2062 at either 2.4×10⁷ PFU/mouse or 2.4×10⁵ PFU/mouse, respectively. All test-group administrations were via intratumoral injections. All animals were appropriately identified by markings on different body parts, and housed and fed according to standard protocols. The longest diameter (mm) and the shortest diameter (mm) of tumors were measured by digital caliper at least 3 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

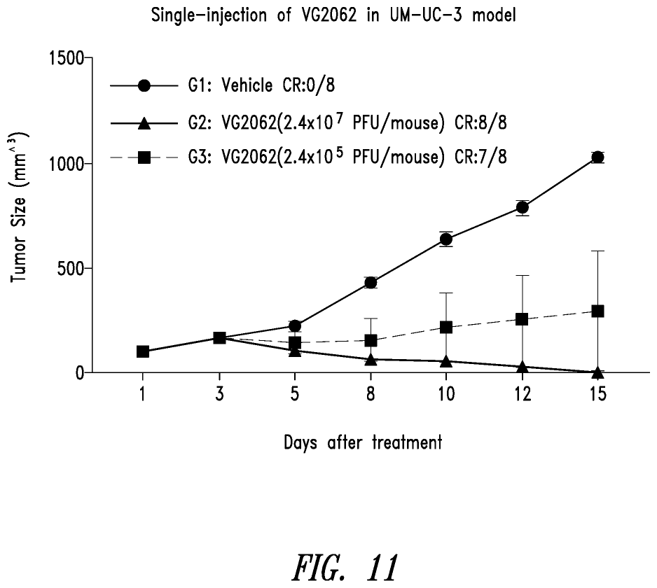

Results: Based on the tumor volume results on day 17, there was a statistically significant difference in tumor volume between the G2 (VG2062, 2.40×107) group and the vehicle control group (P=0.000) (FIG. 11). All 8 of the mice in Group 2 and 7 of the mice in Group 3 had complete tumor regression on day 17, while none of the animals in Group 1 exhibited tumor regression. VG2062 was well tolerated by the mice during this study, and these data indicate that VG2062 may have a significant anti-tumor effect in bladder cancer cells such as UM-UC-3.

### Example 12

### Evaluation of VG2062 Efficacy in the BT-474 Human Breast Cancer Xenograft Model

Objective: CXCR4 has been widely implicated in the progression of breast cancer and other malignancies. CXCR4 expression in breast cancer is associated with aggressiveness and a poor prognosis. The objective of this study was to evaluate the anti-tumor efficacy of VG2062 administered intratumorally (i.t.) in the BT-474 human breast cancer xenograft model in female BALB/c nude mice.

Procedure: 24 female BALB/c nude mice were subcutaneously implanted with BT-474 cells. Mice were randomized into 3 groups, with 8 mice in each group. The day of randomization was defined as day 0. Group 1 was administered DPBS+7.5% glycerin (vehicle control). Groups 2 and 3 were test groups receiving multiple doses of VG2062 (0.1 mL/animal) at two different dose levels via intratumoral injection. Group 2 received 3 doses of 1×10⁵ PFU/mouse/dose at days 1, 2, and 3. Group 3 received 3 doses of 1×10⁷ PFU/mouse/dose at days 1, 2, and 3. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

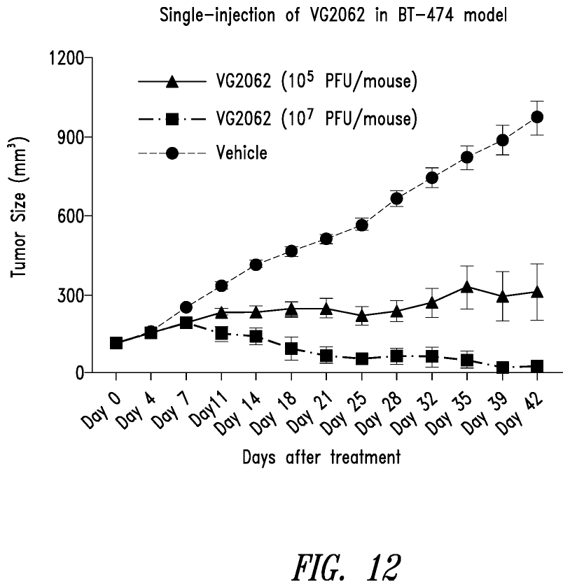

Results: There was no statistical difference in tumor volume at the time of randomization (Day 0). During the experiment, the tumor volumes of the vehicle control group (Group 1) mice continued to increase, while tumor volumes in mice treated with 3 doses of 1×10⁵ PFU/mouse/dose and 3 doses of 1×10⁷ PFU/mouse/dose of VG2062 remained relatively stable and decreased, respectively (FIG. 12). Compared to the vehicle control group, groups 2 and 3 both showed statistically significant inhibition of tumor growth (P<0.05). VG2062 was well tolerated by the mice during this study, and these data indicate that VG2062 may have a significant anti-tumor effect in breast cancer cells such as BT-474.

### Example 13

Treatment of Breast Cancer EMT-6-Bearing Mice with mVG2031

Objective: To evaluate the effect of mVG2031 on inhibiting the growth of the murine breast cancer cell line EMT-6 in immunocompetent BALB/c mice after subcutaneous implantation. mVG2031 is the murine version of VG2062, in which the human IL-12 is replaced by murine IL-12.

Procedure: 24 female immunocompetent BALB/c mice were subcutaneously implanted with EMT-6 cells. Mice were randomized into 3 groups, with 8 mice in each group. The day of randomization was defined as day 0. Group 1 was administered DPBS+7.5% glycerin (vehicle control). Groups 2 and 3 were test groups receiving multiple doses of mVG2031 (0.1 mL/animal) at two different dose levels via intratumoral (i.t.) injection. Group 2 received 3 doses of 1×10⁶ PFU/mouse/dose at days 1, 2, and 3. Group 3 received 3 doses of 1×10⁷ PFU/mouse/dose at days 1, 2, and 3. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

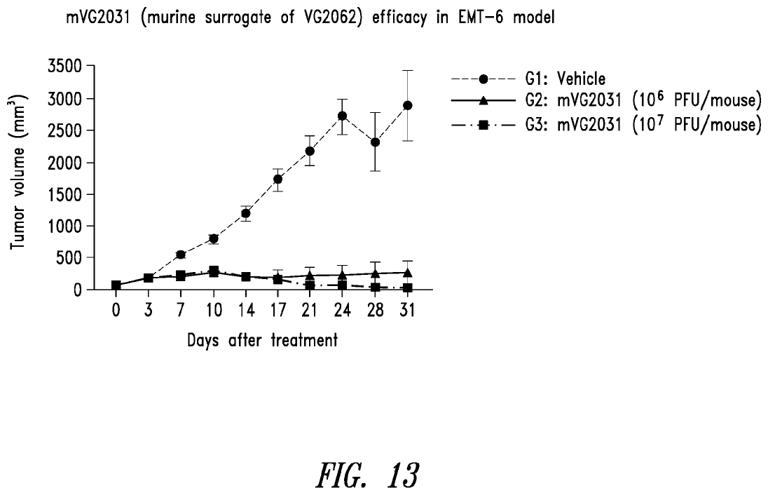

Results: There was no statistical difference in tumor volume at the time of randomization (Day 0). During the experiment, the tumor volumes of the vehicle control group (Group 1) mice continued to increase, while tumor volumes in mice treated with 3 doses of 1×10⁶ PFU/mouse/dose and 3 doses of 1×10⁷ PFU/mouse/dose of mVG2031 remained relatively stable (FIG. 13). Compared to the vehicle control group, groups 2 and 3 both showed statistically significant inhibition of tumor growth (P<0.05). mVG2031 was well tolerated by the mice during this study, and these data indicate that mVG2031 may have a significant anti-tumor effect in breast cancer cells such as EMT-6.

### Example 14

Treatment of Breast Cancer EMT-6-Bearing Mice with a Combination of mVG2031 and Anti-PD-1

Objective: Co-administration of OVs with checkpoint inhibitors is an emerging treatment modality that shows promise for enhancing the efficacy of oncolytic virotherapy. This study sought to evaluate the effect of combining mVG2031 with a checkpoint inhibitor (anti-PD-1 antibody) on inhibiting the growth of the murine breast cancer cell line EMT-6 in immunocompetent BALB/c mice after subcutaneous implantation. mVG2031 is the murine version of VG2062, in which the human IL-12 is replaced by murine IL-12.

Procedure: 32 female immunocompetent BALB/c mice were subcutaneously implanted with EMT-6 cells. Mice were randomized into 4 groups, with 8 mice in each group. The day of randomization was defined as day 0. The first group was intratumorally administered DPBS+7.5% glycerin (vehicle control). The second group was intratumorally treated with 3 doses of mVG2031(1×10⁶ PFU/mouse/dose) at days 1, 2, and 3. The third group was injected intraperitoneally with anti-PD-1 antibody at 10 mg/kg biweekly. The fourth (combination treatment) group was intratumorally treated with 3 doses of mVG2031 (1×10⁶ PFU/mouse/dose) at days 1, 2, and 3 and also injected intraperitoneally with anti-PD-1 antibody at 10 mg/kg biweekly. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

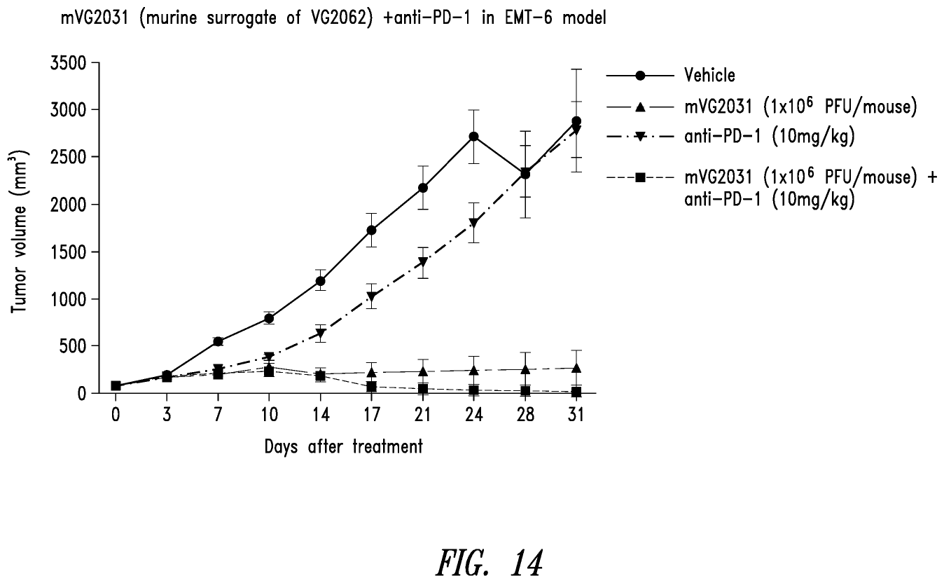

Results: There was no statistical difference in tumor volume at the time of randomization (Day 0). During the experiment, the tumor volumes of both control groups lacking virus (Vehicle and anti-PD-1 alone) continued to increase, while tumor volumes in mice treated with mVG2031 (both with and without co-administration of anti-PD-1) decreased dramatically (FIG. 14). Compared to the non-virus-treated control groups, both virus-treated groups showed statistically significant inhibition of tumor growth (P<0.05). Notably, the combination of virus+anti-PD-1 yielded a stronger anti-tumor effect than treatment with virus alone, as evidenced by almost complete tumor remission in animals treated with the combination of virus and checkpoint inhibitor. mVG2031 was well tolerated by the mice during this study, and these data suggest that combining mVG2031 with a checkpoint inhibitor such as anti-PD-1 antibody may have a significant anti-tumor effect in breast cancer cells such as EMT-6.

### Example 15

Treatment of Renal Cancer Renca-Bearing Mice with mVG2031

Objective: To evaluate the effect of mVG2031 on inhibiting the growth of the murine renal cancer cell line Renca in immunocompetent BALB/c mice after subcutaneous implantation. mVG2031 is the murine version of VG2062, in which the human IL-12 is replaced by murine IL-12.

Procedure: 50 SPF-grade female nude mice (5-6-weeks-old) were subcutaneously implanted with 1×10⁶ Renca cells/mouse. The inoculation site was the right side of the mouse's back near the armpit. Tumor growth was monitored, and when tumors grew to an average volume of about 100-120 mm³, 25 tumor-bearing mice were randomly divided into 5 groups of 5 mice according to tumor volume and body weight. The day of group dosing was defined as day 0. Group 5 was administered DPBS+7.5% glycerin (vehicle control). Groups 1 to 4 were test groups receiving either one or three doses of mVG2031 at two different dose levels via intratumoral injection. Group 1 received a single dose of 5.2×10⁵ PFU/mouse at day 0. Group 2 received a single dose of 5.2×10⁷ PFU/mouse at day 0. Group 3 received 3 doses of 5.2×10⁵ PFU/mouse at days 0, 1, and 2. Group 4 received 3 doses of 5.2×10⁷ PFU/mouse at days 0, 1, and 2. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

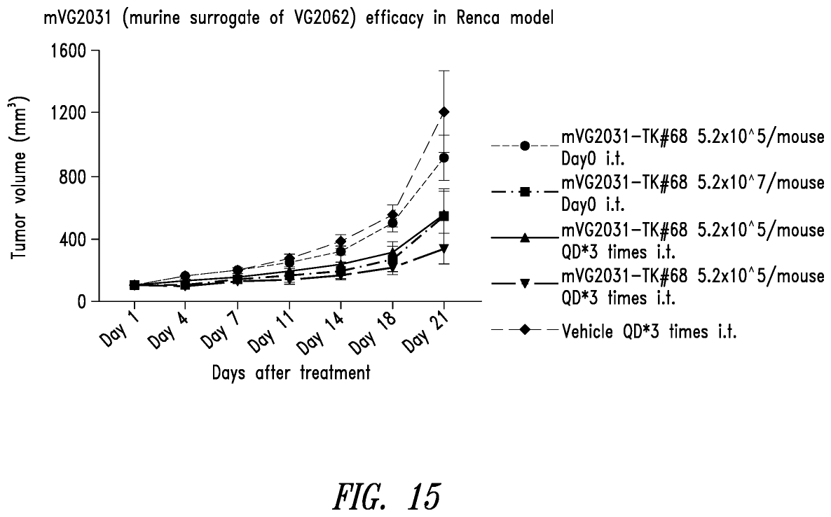

Results: During the experiment, there was no statistical difference in tumor volume on day 0. The tumor volumes of the vehicle control group (Group 5) continued to increase, and on day 21 the tumor volume of Group 5 reached 1204.23 t 253.76 mm³ (FIG. 15). This indicates that the mouse renal cancer cell line Renca was successfully established in the subcutaneous transplanted tumor model using BALB/c mice. mVG2031 was also shown to inhibit the growth of tumors in the Renca model dose-dependently. In Group 1 (5.2×10⁵ PFU/animal, single dose), the tumor volume reached 911.45±143.31 mm³ on day 21. In Group 2 (5.2×10⁷ PFU/animal, single dose), the tumor volume reached 542.27±174.45 mm³ on day 21. In Group 3 (5.2×10⁵ PFU/animal, 3 doses), the tumor volume reached 567.49±133.36 mm³ on day 21. In Group 4 (5.2×10⁷ PFU/animal, 3 doses), the tumor volume reached 337.03±95.93 mm³ on day 21. There was no statistically significant difference in tumor volume in Group 1, but the decrease in tumor volume observed in Groups 2, 3, and 4 passed the threshold for statistical significance (P<0.05). These data indicate that mVG2031 may have a significant anti-tumor effect in renal cancer cells such as Renca.

### Example 16

Treatment of Bilateral Renal Cancer Renca-Bearing Mice with a Combination of mVG2031 and Anti-PD-1

Objective: Co-administration of OVs with checkpoint inhibitors is an emerging treatment modality that shows promise for enhancing the efficacy of oncolytic virotherapy. This study sought to evaluate the effect of combining mVG2031 with a checkpoint inhibitor (anti-PD-1 antibody) on inhibiting the growth of the murine renal cancer cell line Renca in immunocompetent BALB/c mice after subcutaneous implantation. A bilateral Renca tumor model was used to evaluate abscopal tumor clearance. mVG2031 is the murine version of VG2062, in which the human IL-12 is replaced by murine IL-12.

Procedure: 24 female immunocompetent BALB/c mice were subcutaneously implanted with Renca cells in both left and right flanks. Mice were randomized into 4 groups, with 6 mice in each group. The day of randomization was defined as day 0. One group was intratumorally administered DPBS+7.5% glycerin (vehicle control) into the right flank tumor. A second group was intraperitoneally administered 10 mg/kg of anti-PD-1 antibody biweekly. The third group was intratumorally injected (into the right side tumor) 3 times with mVG2031 (5×10⁷ PFU/mouse/dose) at days 1, 2, and 3. The fourth group was likewise intratumorally injected (into the right side tumor) 3 times with mVG2031 (5×10⁷ PFU/mouse/dose) at days 1, 2, and 3, and also intraperitoneally administered 10 mg/kg of anti-PD-1 antibody biweekly. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

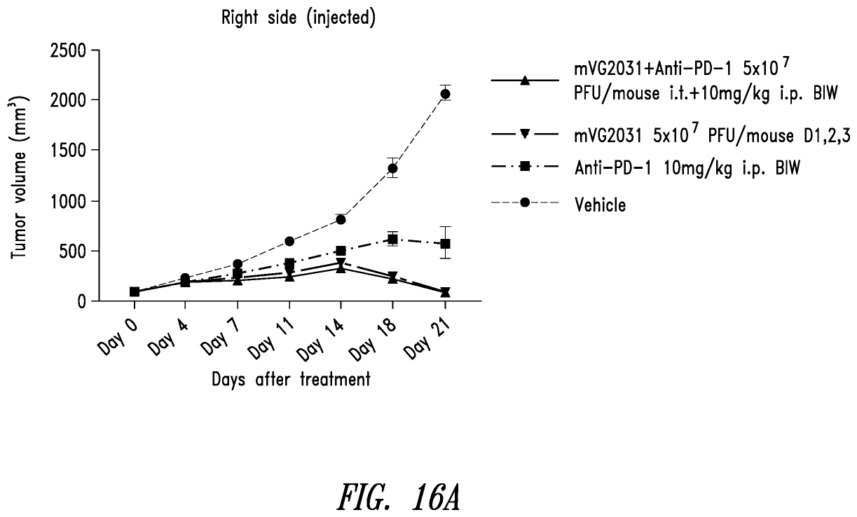

Results: There was no statistical difference in tumor volume on both flanks at the time of randomization (Day 0). During the experiment, all tumor volumes on the non-injected side and the tumor volumes of both control groups lacking virus on the injected side (Vehicle group and anti-PD-1 group) continued to increase, although tumors on the injected side that were treated with anti-PD-1 alone remained significantly smaller than tumors treated with vehicle control (FIGS. 16A and 16B). Tumor volumes on the injected side in mice treated with mVG2031 (both with and without co-administration of anti-PD-1) decreased dramatically. Compared to the non-virus-treated control groups, both groups that were treated with mVG2031 showed statistically significant inhibition of tumor growth (P<0.05) on the injected side. Only the group treated with both mVG2031 and anti-PD-1 reached statistical significance for tumor reduction on the non-injected side when compared to mice treated with vehicle control. mVG2031 was well tolerated by the mice during this study, and these data suggest that combining mVG2031 with a checkpoint inhibitor such as anti-PD-1 antibody may have a significant anti-tumor effect in renal cancer cells such as Renca.

### Example 17

### Treatment of Renal Cancer A498-Bearing Nude Mice

Objective: To evaluate the anti-tumor activity of VG2062 in a human renal cancer A498 cell line-derived xenograft (CDX) model.

Procedure: 24 SPF-grade female nude mice were subcutaneously implanted with 1×10⁷ A498 cells/mouse and randomized into 3 groups, with 8 mice in each group. The day of randomization was noted as day 0. Group 1 was administered DPBS+7.5% glycerin (vehicle control). Groups 2 and 3 were low and high dose test groups receiving multiple doses of VG2062 at either 2.4×10⁵ PFU/mouse/dose or 2.4×10⁷ PFU/mouse/dose, respectively. Doses were administered at day 0, day 7, day 10, day 11, and day 12. All test-group administrations were via intratumoral injections. All animals were appropriately identified by markings on different body parts, and housed and fed according to standard protocols. The longest diameter (mm) and the shortest diameter (mm) of tumors were measured by digital caliper at least 2 times/week. The tumor volume was calculated as TV=0.5×a×b², where: TV=tumor volume, mm³; a=longest diameter, mm; b=shortest diameter, mm. The average tumor volume of each group was calculated and represented as mean±standard error of mean (SEM). P value of less than 0.05 was considered to be statistically significant.

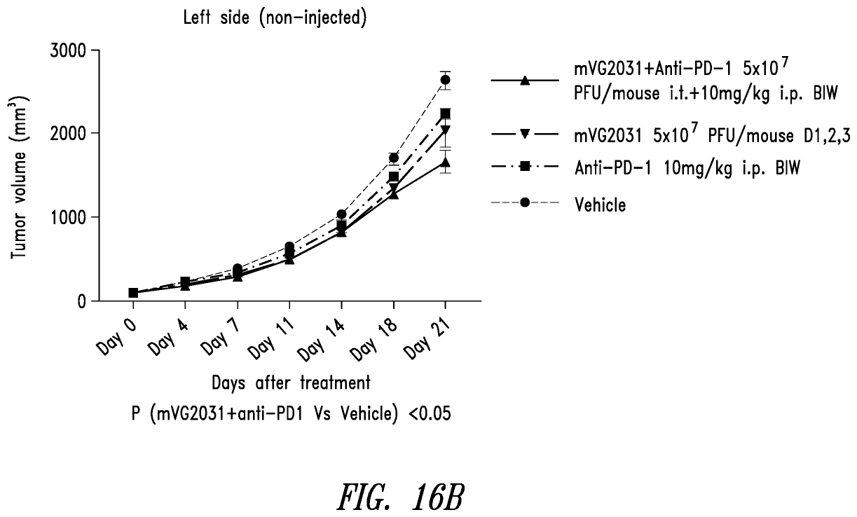

Results: The experiment was completed on the 21st day after the first treatment administration, and the mean tumor volume of the tumor-bearing mice in the vehicle control group was recorded as 1873.58±261.30 mm³. Tumor volumes in the VG2062 high-dose group and the VG2062 low-dose group were 1068.56±157.43 mm³ and 1284.69±108.84 mm³, respectively (FIG. 17). Compared with the vehicle control group, each virus-treated group showed statistically significant inhibition of tumor growth (P<0.05). VG2062 was well tolerated by the mice during this study, and these data indicate that VG2062 may have a significant anti-tumor effect in renal cancer cells such as A498.

### Example 18

### Corneal Inoculation in BALB/c Mouse Model

Objective: To evaluate VG2062 ocular pathogenicity after corneal inoculation in a BALB/c mouse model.

Procedure: SPF-grade, 8-week-old female BALB/c mice were randomized into 4 groups. Mice were anesthetized by injecting 10 mg of ketamine hydrochloride per 100 g of body weight into the hind leg muscles. The left and right cornea were lightly scarified with an insulin needle to avoid breaking the cornea. 5 μl of virus solution was administered into the scarified left and right eyes. One group received 10⁵ PFU/mouse of VG2062, another group received 10⁷ PFU/mouse of VG2062, the third group received 10⁵ PFU/mouse of wild-type HSV-1 control strain 17+, and the fourth group received vehicle control consisting of Vero cell supernatant (5 μl/eye).

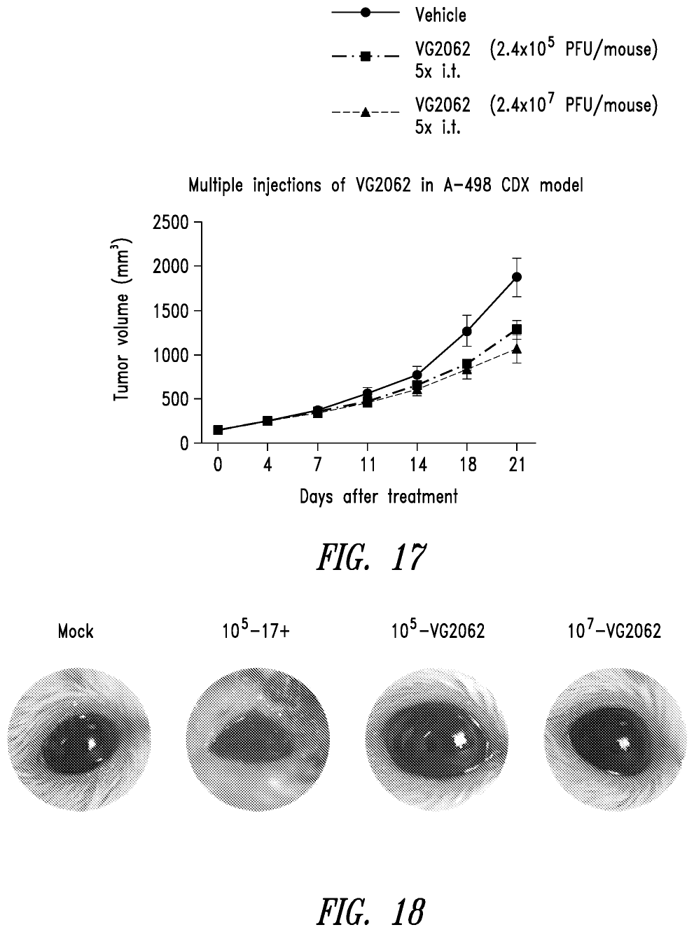

Results: Corneal scarification is an effective way of establishing HSV-1 infection. In this study, inoculation with 10⁵ PFU/mouse of wild-type HSV-1 17+ led to severe ocular inflammation, including difficulty in opening eyes and hair loss around the eyes. By contrast, the VG2062-infected mice exhibited minimal eye disease even at the much higher dosage of 107 PFU/mouse, with no significant differences in eye pathology compared to mice treated with vehicle control (FIG. 18).

The following are some exemplary numbered embodiments of the present disclosure.

1. A recombinant herpes simplex virus comprising a modified oncolytic herpes virus genome, wherein the modified herpes virus genome comprises at least one miRNA target sequence operably linked to a first copy of an ICP34.5 gene, and a second copy of the ICP34.5 gene comprises an inactivating mutation, and further comprising replacing an ICP47 promoter with an immediate early gene promoter such as an ICP4, ICP0, or, ICP27 regulatory sequence operably linked to an ICP47 gene, and wherein the ICP47 gene comprises an inactivating deletion in the natural ICP47 regulatory sequence. Within further embodiments, the immediate early gene promoter may be selected from either HSV-1 or HSV-2.

2. The recombinant herpes simplex virus of embodiment 1, further comprising a deletion of one RL and one RS region of the viral genome. Within optional embodiments the mutation is a deletion containing the second copy of the ICP34.5 gene.

3. The recombinant herpes simplex virus according to any one of embodiments 1 or 2, comprising from two to ten miRNA target sequences operably linked to the first copy of the ICP34.5 gene. Within a further embodiments the miRNA target sequences are inserted in tandem into the 3′ untranslated region. Within various embodiments, identical, or, varying lengths of linker DNA can be inserted between different miRNA binding sites. Within certain embodiments the linkers range from 1 to 50 base pairs. Within other embodiments the linker is less than 10 base pairs.

4. The recombinant herpes simplex virus according to any one of embodiments 1, 2, or 3, wherein the miRNA target sequences are inserted into a 3′ untranslated region of the first copy of the ICP34.5 gene.

5. The recombinant herpes simplex virus according to any one of embodiments 1, 2, 3 or 4, wherein the from two to ten miRNA target sequences bind at least two different miRNAs.

6. The recombinant herpes simplex virus according to any one of embodiments 1, 2, 3, 4, or 5, wherein the miRNAs are selected from the group consisting of miR-124, miR-124*, and miR-143.

7. The recombinant herpes simplex virus according to any one of embodiments 1, 2, 3, 4, 5, or 6, wherein the modified herpes virus genome comprises additional mutations or modifications in viral genes ICP0, ICP4 and/or ICP27. Within further embodiments, the mutations or modifications can comprise a deletion of one copy of either RL or RS.

8. The recombinant herpes simplex virus according to any one of embodiments 1, 2, 3, 4, 5, 6, or 7, wherein the modification comprises replacing a native viral promoter with a tumor specific promoter.

9. The recombinant herpes simplex virus according to any one of embodiments 1, 2, 3, 4, 5, 6, 7, or 8, wherein the modification is replacement of the entire promoter-regulatory region of ICP27, optionally, with a tumor specific promoter.

10. The recombinant herpes simplex virus according to any one of embodiments, 1, 2, 3, 4, 5, 6, 7, 8, or 9, wherein the ICP27 promoter is replaced with a CXCR4 promoter.

11. The recombinant herpes simplex virus according to any one of embodiments 1, 2, 3, 4, 5, 6, 7, 8, 9, or 10, further comprising at least one nucleic acid encoding a non-viral protein selected from the group consisting of immunostimulatory factors, antibodies, and checkpoint blocking peptides, wherein the at least one nucleic acid is operably linked to a generic, or, a tumor-specific promoter. Examples of generic promoters include constitutive promoters such as SV40, CMV, UBC, EF1alpha, PGK and CAGG.

12. The recombinant herpes simplex virus according to any one of embodiments, 1, 2, 3, 4, 5, 6, 7, 8, 9, or 11, wherein the non-viral protein is IL12, IL15, an IL15 receptor alpha subunit, a CTLA-4 antagonist, PD-1 antagonist, PD-L1 antagonist and/or PD-L2 antagonist. Within certain embodiments the Herpes simplex viruses encodes IL12, IL15, an IL15 receptor alpha subunit, and a PD-1 antagonist such as Pembrolizumab.

13. The recombinant herpes simplex virus according to any one of embodiments 11 or 12, wherein the promoter is a CMV promoter.

14. The recombinant herpes simplex virus according to any one of embodiments 1, 2, 3, 4, 5, 6, 7, 8, 9, 10, 11, 12, or 13 having a nucleic acid sequence encoding a glycoprotein with enhanced fusogenicity (as compared to a similar wild-type virus). Examples include a wide variety of transgenes (e.g., a fusogenic glycoprotein from Gibbon Ape Leukemia Virus “GALV”), and/or mutations which enhance HSV fusion, including for example, a truncations or mutations in glycoprotein B, glycoprotein K, and or UL20. Within a preferred embodiment the nucleic acid sequence encodes a fusogenic form of glycoprotein B (e.g., glycoprotein B which is truncated after amino acid 876). Within further embodiments, replacing the ICP47 promoter with an ICP27 promoter can be utilized to increase fusogenicity.

15. The recombinant herpes simplex virus according to any one of embodiment 14, wherein the glycoprotein B can be truncated with a deletion occurring after amino acid 876.

16. The recombinant herpes simplex virus according to any one of embodiments 1, 2, 3, 4, 5, 6, 7, 8, 9, 10, 11, 12, 13, 14, or 15, wherein the oncolytic herpes virus is HSV-1. Within particularly preferred embodiments of the invention the recombinant herpes simplex virus comprises an oncolytic HSV-1 wherein: a) there is a deletion of one RL containing the genes encoding ICP0 and ICP34.5 and a deletion of one RS containing the gene encoding ICP4; b) replacement of a native ICP27 promoter with a CXCR4 promoter; c) insertion of binding sites for miR-143 and miR-124 in the ICP34.5 3′ UTR; d) deletion of a portion of the 3′ end of glycoprotein B coding region (e.g., a 84 bp deletion); e) insertion of an expression cassette which can express L-12, IL-15, and IL-15Rα under the control of a CMV promoter; and f) replacement of a native ICP47 regulatory sequence with an ICP27 regulatory sequence.
wherein the mutation is deletion of one RL and one RS region of the viral genome. Within optional embodiments the mutation is a deletion containing the second copy of the ICP34.5 gene.

17. A method for inhibiting or lysing tumor cells, comprising providing a therapeutically effective amount of recombinant herpes simplex virus according to any one of embodiments 1 to 16.

18. A method for inhibiting tumor cells, comprising providing a therapeutically effective amount of recombinant herpes simplex virus according to any one of embodiments 1 to 17 to tumor cells.

19. The method according to embodiment 18, further comprising the step of providing a CTLA-4, PD-1, PD-L1 and/or PD-L2 antagonist to said cells. Within certain embodiments the antagonist is selected from the group consisting of Ipilimumab, Tremelimumab, Pembrolizumab, Cemiplimab, Dostarlimab, Vopratelimab, Spartalizumab and Camrelizumab.

20. A therapeutic composition comprising the recombinant herpes simplex virus according to any one of embodiments 1 to 17 and a pharmaceutically acceptable carrier.

21. The therapeutic composition according to embodiment 20, further comprising a CTLA-4, PD-1, PD-L1 and/or PD-L2 antagonist. Within certain embodiments the antagonist is selected from the group consisting of Ipilimumab, Tremelimumab, Pembrolizumab, Cemiplimab, Dostarlimab, Vopratelimab, Spartalizumab and Camrelizumab.

22. A method for treating cancer in a subject suffering therefrom, comprising the step of administering a therapeutically effective amount of the composition of embodiment 20.

23. The method according to embodiment 22 wherein said cancer expresses a high level of a biomarker such as CXCR4. Within further embodiments, said cancer expresses a high level of a biomarker, the promoter of which is used to drive ICP4 and/or ICP27 genes according to one of the preceding embodiments. Within other embodiments, the cancer expresses a high level of a biomarker such as, for example, CEA or, CXCR4. Within certain embodiments, the cancer is selected from the group consisting of cancers of the urological system, cervix, esophagus, lung, colorectum, stomach, cholangiocarcinoma and pancreas. Within other embodiments the cancer is selected from the group consisting of breast and prostate tumors, and glioblastomas. Within other embodiments the cancer is a leukemia or a lymphoma. Within other embodiments the cancer is an acute myeloid leukemia (AML) or a B cell lymphoma. Within other embodiments, the cancer is a surface injectable tumor. Within yet other embodiments the cancer expresses a high level of CXCR4.

24. The method according to embodiment 22 wherein said cancer is a urological cancer.

25. The method according to embodiment 22, further comprising the step of administering a CTLA-4, PD-1, PD-L1 and/or PD-L2 antagonist to said subject. Within certain embodiments the antagonist is selected from the group consisting of Ipilimumab, Tremelimumab, Pembrolizumab, Cemiplimab, Dostarlimab, Vopratelimab, Spartalizumab and Camrelizumab.

26. The method according to embodiment 19 wherein said cancer expresses a high level of a biomarker, the promoter of which is used to drive ICP4 and/or ICP27 genes according to one of the preceding embodiments. Within other embodiments, the cancer expresses a high level of a biomarker such as, for example, CEA or, CXCR4. Within certain embodiments, the cancer is selected from the group consisting of cancers of the urological system, cervix, esophagus, lung, colorectum, stomach, cholangiocarcinoma and pancreas. Within other embodiments the cancer is selected from the group consisting of breast and prostate tumors, and glioblastomas. Within other embodiments the cancer is a leukemia or a lymphoma. Within other embodiments the cancer is an acute myeloid leukemia (AML) or a B cell lymphoma. Within other embodiments, the cancer is a surface injectable tumor. Within yet other embodiments the cancer expresses a high level of CXCR4.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

It is to be understood that the terminology used herein is for the purpose of describing specific embodiments only and is not intended to be limiting. It is further to be understood that unless specifically defined herein, the terminology used herein is to be given its traditional meaning as known in the relevant art.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

Furthermore, the written description portion of this patent includes all claims. Furthermore, all claims, including all original claims as well as all claims from any and all priority documents, are hereby incorporated by reference in their entirety into the written description portion of the specification, and Applicants reserve the right to physically incorporate into the written description or any other portion of the application, any and all such claims. Thus, for example, under no circumstances may the patent be interpreted as allegedly not providing a written description for a claim on the assertion that the precise wording of the claim is not set forth in haec verba in written description portion of the patent.

The claims will be interpreted according to law. However, and notwithstanding the alleged or perceived ease or difficulty of interpreting any claim or portion thereof, under no circumstances may any adjustment or amendment of a claim or any portion thereof during prosecution of the application or applications leading to this patent be interpreted as having forfeited any right to any and all equivalents thereof that do not form a part of the prior art.

Other nonlimiting embodiments are within the following claims. The patent may not be interpreted to be limited to the specific examples or nonlimiting embodiments or methods specifically and/or expressly disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

What is claimed is:

1. A recombinant herpes simplex virus comprising a modified oncolytic herpes virus genome, wherein the modified herpes virus genome comprises at least one miRNA target sequence operably linked to a first copy of an ICP34.5 gene, and a second copy of the ICP34.5 gene comprises an inactivating mutation, and further comprising replacing an ICP47 promoter with an immediate early gene promoter such as an ICP4, ICP0, or, ICP27 regulatory sequence operably linked to an ICP47 gene, and wherein the ICP47 gene comprises an inactivating deletion in the natural ICP47 regulatory sequence. Within further embodiments, the immediate early gene promoter may be selected from either HSV-1 or HSV-2.

2. The recombinant herpes simplex virus of claim 1, further comprising a deletion of one RL and one RS region of the viral genome. Within optional embodiments the mutation is a deletion containing the second copy of the ICP34.5 gene.

3. The recombinant herpes simplex virus of claim 1, comprising from two to ten miRNA target sequences operably linked to the first copy of the ICP34.5 gene.

4. The recombinant herpes simplex virus of claim 3, wherein the miRNA target sequences are inserted into a 3 untranslated region of the first copy of the ICP34.5 gene.

5. The recombinant herpes simplex virus of claim 3, wherein the from two to ten miRNA target sequences bind at least two different miRNAs.

6. The recombinant herpes simplex virus of claim 5, wherein the miRNAs are selected from the group consisting of miR-124, miR-124*, and miR-143.

7. The recombinant herpes simplex virus of claim 1, wherein the modified herpes virus genome comprises additional mutations or modifications in viral genes ICP0, ICP4 and/or ICP27.

8. The recombinant herpes simplex virus of claim 1, wherein said virus is modified by replacing a native viral promoter with a tumor specific promoter.

9. The recombinant herpes simplex virus of claim 1, wherein the modification is replacement of the entire promoter-regulatory region of ICP27 with a tumor specific promoter.

10. The recombinant herpes simplex virus of claim 9, wherein the ICP27 promoter is replaced with a CXCR4 promoter.

11. The recombinant herpes simplex virus of claim 7, wherein the modification is deletion of one copy of either RL or RS.

12. The recombinant herpes simplex virus of claim 1, further comprising at least one nucleic acid encoding a non-viral protein selected from the group consisting of immunostimulatory factors, antibodies, and checkpoint blocking peptides, wherein the at least one nucleic acid is operably linked to a generic or a tumor-specific promoter.

13. The recombinant herpes simplex virus of claim 11, wherein the non-viral protein is IL12, IL15, IL15 receptor alpha subunit, a CTLA-4 antagonist, a PD-1 antagonist, a PD-L1 antagonist, and/or a PD-L2 antagonist.

14. The recombinant herpes simplex virus of claim 12, wherein the generic promoter is CMV.

15. The recombinant herpes simplex virus of claim 1, further comprising a nucleic acid sequence encoding a fusogenic form of glycoprotein B.

16. The recombinant herpes simplex virus of claim 14, wherein the glycoprotein B can be truncated with a deletion occurring after amino acid 876.

17. The recombinant herpes simplex virus of any one of claims 1 to 15, wherein the oncolytic herpes virus is HSV-1.

18. A method for inhibiting tumor cells, comprising providing a therapeutically effective amount of recombinant herpes simplex virus according to any one of claims 1 to 17 to tumor cells.

19. The method according to claim 18, further comprising the step of providing a CTLA-4, PD-1, PD-L1 and/or PD-L2 antagonist to said cells.

20. A therapeutic composition comprising the recombinant herpes simplex virus according to any one of claims 1 to 17 and a pharmaceutically acceptable carrier.

21. The therapeutic composition according to claim 20, further comprising a CTLA-4, PD-1, PD-L1 and/or PD-L2 antagonist.

22. A method for treating cancer in a subject suffering therefrom, comprising the step of administering a therapeutically effective amount of the composition of claim 20.

23. The method according to claim 22 wherein said cancer expresses a high level of a biomarker such as CXCR4.

24. The method according to claim 22 wherein said cancer is a urological cancer.

25. The method according to claim 22, further comprising the step of administering a CTLA-4, PD-1, PD-L1 and/or PD-L2 antagonist to said subject.
